(19) United States
(12) Patent Application Publication
(10) Pub. No.: US 2009/0156637 A1
(43) Pub. Date: Jun. 18, 2009

(54) BUTYROPHENONES AND SIGMA-1 RECEPTOR ANTAGONISTS PROTECT AGAINST OXIDATIVE-STRESS

(75) Inventors: John A. Schetz, Fort Worth, TX (US); James Simpkins, Fort Worth, TX (US); Jeffery Talbert, Austin, TX (US)

Correspondence Address: CHALKER FLORES, LLP, 2711 LBJ FRWY, Suite 1036, DALLAS, TX, 75234, US

(73) Assignee: University of North Texas Health Science Center of Fort Worth, Fort Worth, TX (US)

(21) Appl. No.: 12/391,141

(22) Filed: Feb. 23, 2009

Related U.S. Application Data
(62) Division of application No. 11/267,030, filed on Nov. 3, 2005.
(60) Provisional application No. 60/624,899, filed on Nov. 3, 2004.

(51) Int. Cl. A61K 31/445; A61P 9/10

(52) U.S. Cl. 514/315

(57) ABSTRACT

The present invention includes compositions and methods for the protection of one or more central nervous system cells from trauma, when administered before, during or after the trauma, wherein the composition includes an effective amount of a butyrophenone, e.g., a 1-linked phenyl butyrophenone that is electronegative along the butyl chain and/or a Sigma-1 receptor antagonist.

This application claims priority to U.S. Provisional Patent Application Ser. No. 60/624,899, filed Nov. 3, 2004, the entire contents of which are incorporated herein by reference. Without limiting the scope of the invention, its background is described in connection with, for example, ischemic cerebral stroke.

### TECHNICAL FIELD OF THE INVENTION

The present invention relates in general to the field of protecting cells from trauma, and more particularly, to compositions and methods for the protection of cells of the central nervous system using butyrophenones and other compounds that antagonize Sigma-1 receptors.

### BACKGROUND OF THE INVENTION

In the United States, brain stroke is the third leading cause of death (Rosenberg et al., 1996). An estimated 80% of strokes are classified as ischemic strokes in which oxygen deprivation results in oxidative stress (OS) (Mohr et al., 1978). Even relatively short durations of OS can trigger cell dysfunction, or worse, cell death (Tan et al., 1998). Age is a major risk factor under conditions of oxidative stress, because youthful homeostatic systems that are generally effective in combating oxidative injury are compromised in aging populations (Droge, 2003; Junqueira et al., 2004). Oxidative stress can be induced by a variety of age-related disorders and insults other than ischemic stroke, including cerebrovascular disease and head trauma, and neurodegenerative diseases with a secondary inflammatory component, such as Alzheimer's disease and Parkinson's disease (Eikelenboom et al., 1998; Perry et al., 2002). Although there are promising preclinical strategies for combating oxidative stress-related brain injury, such as the use of non-feminizing estrogens and various antioxidants (Liu et al., 2002; Bhavnani, 2003; Calabrese et al. 2003; Granot and Kohen, 2004), there is a remarkable dearth of pharmacotherapies currently in clinical use.

The positive correlation between the number of traumatic brain injuries (for any reason) and increased risk for developing the most prevalent, sporadic form of Alzheimer's disease (AD) later in life (Plassman et al., 2000; Fleminger et al., 2003) has lead some to propose a mechanistic link between the brain damage due to ischemic cerebral vascular events, including head trauma, stroke and epilepsy (de al Torre, 2004; Eikelenboom, et al., 1998; Mortimer et al., 1985; Mortimer et al., 1991; van Duijn et al., 1992; Guo et al., 2000; Fleminger et al, 2003; Honig et al., 2004), and neurodegenerative disorders like AD (Stepanichev et al., 1998), because all these conditions eventually result in the production of free radicals that induce oxidative damage (Aliev et al., 2002; Aliev et al., 2003). The burgeoning idea that antipsychotic drugs might be neuroprotective in schizophrenia (Nisenbaum et al., 2003; Dichter and Locke, 2003; Berger et al., 2003) coupled with the relatively common practice of treating the psychotic and agitation/aggression symptoms in AD with antipsychotics (Devanand et al., 1998; Salzman, 2001; Pelton et al., 2003; Mintzer and Targum, 2003) led to the investigation of whether antipsychotics, in addition to their palliative role in treating agitation associated with AD, might also serve a neuroprotective role by preventing brain neurodegeneration in response to toxic insults.

### SUMMARY OF THE INVENTION

The present invention includes compositions and methods for the use of butyrophenones (e.g., the class of compounds having the basic core structure 4-[4-(Aryl)-4-hydroxy-1-piperidyl]-1-(Ary1)-butan-1-one) and other non-butyrophenone Sigma-1 antagonists as protective agents against oxidative stress related brain traumas. Specific butyrophenone substructural features that correlate well with protection in the initial data set are those with a 1-linked phenyl and an electronegative moiety (e.g., keto or hydroxyl) at the 1 position of the butyl chain.

Examples of such drugs that are currently approved for the treatment of other indications such as psychosis or deviant sexual behavior (e.g., USA, Europe or Asia) are haloperidol, bromperidol, penfluridol and trifluperidol. Additional compounds with the same structural motif are chlorinated haloperidol and the haloperidol metabolite II (the butyl keto is reduced to a hydroxyl). A wide range of potential clinical applications are plausible due to the belief that a diverse range of events lead to oxidative stress (e.g., head trauma, ischemic stroke, neurodegenerative diseases such as Alzheimer's disease and Parkinson's disease, and neuropsychiatric disorders, like schizophrenia and depression), and epilepsy and brain infections. Protection against ischemic cerebral stroke was the model of oxidative stress related brain damage, because transient middle cerebral artery (tMCA) occlusion methods can be applied to rats that mimic the early phase ischemic events in stroke patients. For example, for haloperidol the oral dose is typically in the range of 2-20 mg/day, but can be as high as 60-100 mg/day in those that are non-responsive at lower doses. The optimal dose range that produces the desired clinical effect with a minimal risk of side-effects is one that results in D2 dopamine receptor occupancy that is between about 65-75% in vivo (Kapur, et al., 2000).

The present invention includes compositions and methods for the protection of one or more cells, e.g., cells of the central nervous system, from ischemic trauma, when administered before, during or after the trauma, e.g., immediately following the trauma. The compositions generally include an effective amount of a butyrophenone, e.g., having a substituted phenyl and that is electronegative along the butyl chain. In one example, the composition of the present invention provides protection from ischemia in a mammalian subject in need thereof comprising a pharmaceutically effective amount of one or more antipsychotic butyrophenones. For example, the butyrophenone may be 1-linked phenyl butyrophenone provided at between about 0.05 and 30 mg per day. The butyrophenone may be any of the 1-linked phenyl butyrophenones, which may include an electronegative moiety at position 4 of the butyl chain. The butyrophenone may be selected from one or more of the following: Haloperidol, Haloperidol decanoate Trifluperidol, Chlorohaloperidol, Bromperidol, Haloperidol metabolite II (Reduced haloperidol), and metabolites thereof. While Penfluridol for not have an electronegative moiety at position 4, it has also been found to be useful in conjunction with the present invention,

One embodiment of the present invention includes providing a patient with an amount of butyrophenone in an amount sufficient to occupy greater than about 65% of the D2 dopamine receptor in vivo. However, in the case of haloperidol, which has about 400-fold less affinity for the D2 receptor than haloperidol, the amount of occupancy required would be reduced greatly. The butyrophenone may be adapted for oral, intravenous, subcutaneous or intramuscular administration. Examples of pharmaceutically effective amounts of the butyrophenone at about 0.01 mg/kg to about 10 mg/kg for 0.5 to 96 hours. The butyrophenone may be adapted for administration to a patient before a surgery that will comprise an ischemic interval, e.g., during a planned surgery that includes a potential for tissues to undergo ischemia for a prolonged period of time.

The present invention also includes a method for reducing the effect of ischemia by contacting cells with a pharmaceutically effective amount of one or more butyrophenones, and/or a sigma-1 receptor antagonists that protect the cells from the ischemia. In one example, the composition is administered several hours before to about 720 minutes after the occurrence of an ischemic cerebral trauma. The ischemic injury may be a cerebral vascular accident, a head trauma or a stroke. The composition may be provided in conjunction with and/or at about the same time as a therapeutic agent selected from the group consisting of t-PA, streptokinase, urokinase, aspirin, dipyridamole, a thrombolytic, an antithrombotic drug, combinations and mixtures thereof. Generally, the one or more butyrophenones are provided at a dose between about 0.5 and 100 mg per day. The butyrophenone for use with the method of the present invention may be selected from one or more of the following: Haloperidol, Haloperidol decanoate, Trifluperidol, Chlorohaloperidol, Bromperidol, Penfluridol, Haloperidol metabolite II (Reduced haloperidol), combinations and metabolites thereof.

The butyrophenone may be a 1-linked phenyl butyrophenone in an amount sufficient to occupy greater than about 65% of the D2 dopamine receptor in vivo and may be adapted for intravenous, subcutaneous, oral, intramuscular or other use. Often, the butyrophenone of the present invention may be provided in or with a pharmaceutically acceptable carrier at, e.g., a pharmacologically effective amount of butyrophenones from between about 0.5 mg/kg to about 30 mg/kg. Generally, the dose may be between 0.5 mg/kg to about 5 mg/kg. Any route of administration for the butyrophenones may be used, e.g., the butyrophenones may be adapted for oral, intravenous, subcutaneous, sublingual, intramuscular, intranasal or mucosal administration. In one embodiment, the composition may be adapted to release at least 90% of the butyrophenones between about 5 and 360 minutes. Alternatively, the composition may be adapted to release at least 90% of the butyrophenones between about 5 minutes and 12 hours. The composition may be packaged into a capsule, caplet, softgel, gelcap, suppository, film, granule, gum, insert, pastille, pellet, troche, lozenge, disk, poultice or wafer.

Yet another embodiment of the present invention includes compositions and methods for reducing the effect of ischemia during surgery by identifying a patient that will undergo an ischemic interval during surgery; and providing the patient a pharmaceutically effective amount of one or more butyrophenones sufficient to protect the patient from the ischemic interval. The composition may be administered between about one hour before the surgery to about 2 weeks after the occurrence of an ischemic cerebral trauma. The ischemic injury may be used for surgery before or after a cerebral vascular accident, a head trauma or a stroke. The composition may be provided alone or in combination with a therapeutic agent selected from the group consisting of t-PA, streptokinase, urokinase, aspirin, dipyridamole, a thrombolytic, an antithrombotic drug, combinations and mixtures thereof. The one or more butyrophenones may be provided at a dose between about 0.05 and 30.0 mg per day. Depending on the needs of the patient, the composition may be provided before, during, after the surgery and combinations thereof. Examples of surgeries that benefit from the present invention include general, orthopedic, spinal, coronary artery bypass grafting (CABG), carotid endarterectomy and aneurysm surgeries.

Yet another embodiment of the present invention is a pharmaceutical composition that protects against ischemic stroke comprising a pharmaceutically effective amount of one or more butyrophenones. In some embodiments the composition that provides protection from ischemia includes a pharmaceutically effective amount of one or more butyrophenones that bind a Sigma-1 receptor.

Yet another embodiment of the ischemic protection of the present invention is a composition that provides protection from ischemia in a mammalian subject in need thereof that includes a pharmaceutically effective amount of one or more compounds selected from Haloperidol, Haloperidol decanoate, Trifluperidol, Chlorohaloperidol, Bromperidol, Penfluridol, Haloperidol metabolite II (Reduced haloperidol), Melperone, L745870, L741742, L741741, BD1063, BD1047, RBI-257, L741742, L741741 and L745870 and metabolites thereof. The compositions may be used in a method for reducing the effect of ischemia by contacting one or more cells and/or tissue with a pharmaceutically effective amount of one or more compounds selected from Haloperidol, Haloperidol decanoate, Trifluperidol, Chlorohaloperidol, Bromperidol, Penfluridol, Haloperidol metabolite II (Reduced haloperidol), Melperone, L745870, L741742, L741741, BD1063, BD1047, RBI-257, L741742, L741741 and L745870 and metabolites thereof in an amount sufficient to protect cells from ischemia.

The present invention also includes compositions and methods of treating a human being suffering from ischemia by administering a therapeutically effective amount of a compound of Formula I:

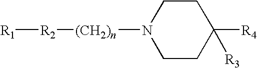

wherein n is 0, 1, 2, 3, 4, 5, or 6; R₁ is a phenyl, a substituted phenyl, a naphthyl, a substituted naphthyl, an indane, a substituted indane, a tetralin, a substituted tetralin, a benzoimidazol, a substituted benzoimidazol, a bisphenyl, a substituted bisphenyl, a benzothiazol, a substituted a benzothiazol; R₂ is C₁₋₆ alkyl, an alcohol or a ketone; R₃ is a hydrogen, a hydroxyl group or an electron pair; (4 is a phenyl, a substituted phenyl, a naphthyl, a substituted naphthyl, an indane, a substituted indane, a tetralin, a substituted tetralin, a benzoimidazol, a substituted benzoimidazol, a benzothiazol, a substituted a benzothiazol, a bisphenyl, a substituted bisphenyl, wherein the substituted groups include hydroxy, alkoxy, alkoxyalkyl, hydroxyl, hydroxyalkyl, alkenyl, amino, nitrate, alkylamino, dialkylamino, nitro, aryl, alkylaryl, arylalkoxy, cycloalkyl, carboxyl, carbonyl, halogen, haloalkyl, haloalkoxy, heteroayl, heterocyclic ring, arylheterocyclic ring, amido, alkylamido, carboxylic ester, carboxylic acid and combinations thereof; and wherein the compound is provided in an amount sufficient to protect cells or tissues from ischemia. Examples of the R₁ group may include one or more chlorophenyls, fluorophenyls and combinations thereof. Examples of the R₄ group may be a chlorophenyl, a bromophenyl, a fluorophenyl, a trichloromethane, a tribromomethane, a trifluoromethane, a dichloromethane, a dibromomethane, a difluoromethane, a chloromethane, a bromomethane or a fluoromethane. The present invention may be used to protect cells, tissue and a patient from the effects of ischemia before, during or after an ischemic event or interval, e.g., cerebral ischemia or a stroke. The ischemia may occur in a tissue that is the subject of a surgical procedure that includes an ischemic event, e.g., general surgery, orthopedic, spinal, coronary artery bypass grafting (CABG), carotid endarterectomy and aneurysms.

In another embodiment, the present invention includes compositions and methods for the treatment of a human being suffering from ischemia by administering a therapeutically effective amount of a 4-[4-(4-chlorophenyl)-4-hydroxy-1-piperidyl]-1-(4-fluorophenyl)-butan-1-one, 1-(4-chlorophenyl)-4-[4-(4-chlorophenyl)-4-hydroxy-1-piperidyl]-butan-1-one, 4-[4-(4-bromophenyl)-4-hydroxy-1-piperidyl]-1-(4-fluorophenyl)-butan-1-one, 1-(4-fluorophenyl)-4-[4-hydroxy-4-[3-(trifluoromethyl)phenyl]-1-piperidyl]-butan-1-one, 1-[1-[4-(4-fluorophenyl)-4-oxo-butyl]-4-piperidyl]-3H-benzoimidazol-2-one, 1-[1-[4-(4-fluorophenyl)-4-oxo-butyl]-3,6-dihydro-2H-pyridin-4-yl]-3H-benzoimidazol-2-one, 8-[4-(4-fluorophenyl)-4-oxo-butyl]-1-phenyl-1,3,8-triazaspiro[4,5]decan-4-one, 1-[4,4-bis(4-fluorophenyl)butyl]-4-[4-chloro-3-(trifluoromethyl)phenyl]-piperidin-4-ol or combinations and mixtures thereof in an amount sufficient to protect a cell or tissue from ischemia.

### BRIEF DESCRIPTION OF THE DRAWINGS

For a more complete understanding of the features and advantages of the present invention, reference is now made to the detailed description of the invention along with the accompanying figures and in which:

FIG. 1 shows the chemical structure of the antipsychotic haloperidol highlighting core and substructural features.

FIG. 2 is a graph that shows an example of some raw data for the in vitro protection assay in a glutamate-induced, oxidative stress-related HT-22 cell model with haloperidol as an example of an antipsychotic drug that provides neuroprotection.

FIG. 3 is a graph that shows raw data for the in vitro protection assay in the glutamate-induced, oxidative stress-related HT-22 cell model with S-(−)-raclopride as an example of an antipsychotic drug that provides no neuroprotection.

FIG. 4 is a graph that shows the [³H]-(+)-pentazocine saturation isotherm binding to a clonal human MCF-7 cell line stably expressing the human Sigma-1 receptor.

FIGS. 5A and 5B are graphs that show a correlational analysis of the potency of in vitro protection and affinity for the cloned Sigma-1 receptor (solid circles represent butyrophenone antipsychotics whose structures are shown in FIG. 6 and open circles represent non-butyrophenone structures), briefly, FIG. 5A: correlation for butyrophenone antipsychotics only; and, FIG. 5B correlation of butyrophenone antipsychotics plus the non-butyrophenone compounds BD1063, L741,742 and L745,870.

FIG. 6 shows the structure-protection relationships of butyrophenones in the in vitro HT-22 cell model of oxidative stress.

FIG. 7 is a graph that shows that Haloperidol is not an antioxidant.

FIG. 8 is a graph that demonstrates the in vivo protection against tMCAO induced brain injury in ovariectomized female Sprague-Dawley rats assessed as infarct volume.

FIG. 9 are representative examples of average images that show that Haloperidol protects against tMCAO brain injury.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “butyrophenone,” “1-linked phenyl butyrophenone” and “1-linked butyrophenone” refer to the class of compounds having the basic core structure 4-[4-(Aryl)-4-hydroxy-1-piperidyl]-1-(Aryl)-butan-1-one. The present invention provides for modifications at any and all of the individual groups of the core structure. For example, 4-chlorophenyl, 4-fluorophenyl, 4-bromophenyl, 3-(trifluoromethyl)phenyl, benzoimidazol-2-one, benzothiazol-2-one and other substitutions known to the skilled artisan may be substituted for the Aryl groups. In some embodiments, the butan-1-one may be reduced to a hydroxyl. Other examples of the butyrophenone include: Haloperidol, Haloperidol decanoate Trifluperidol, Chlorohaloperidol, Bromperidol, Haloperidol metabolite II (Reduced haloperidol), and metabolites thereof. In other embodiments, Aryl and piperidyl may form a bicyclic structure, e.g., 1,3,8-triazaspiro[4,5]decan-4-one. An example of such a compound includes 8-[4-(4-fluorophenyl)-4-oxo-butyl]-1-phenyl-1,3,8-triazaspiro[4,5]decan-4-one. Furthermore, the Aryl may include 2 ring structures, e.g., 4,4-bis(Aryl)butyl. One example of such compounds include 1-[4,4-bis(4-fluorophenyl)butyl]-4-[4-chloro-3-(trifluoromethyl)phenyl]-piperidin-4-ol.

Examples of the compounds that may be provided at regular or sub-optimal doses as antipsychotics, may be provided in lower doses and be effective to protects cells or tissues from ischemic intervals or events and may include e.g., a 4-[4-(4-chlorophenyl)-4-hydroxy-1-piperidyl]-1-(4-fluorophenyl)-butan-1-one, 1-(4-chlorophenyl)-4-[4-(4-chlorophenyl)-4-hydroxy-1-piperidyl]-butan-1-one, 4-[4-(4-bromophenyl)-4-hydroxy-1-piperidyl]-1-(4-fluorophenyl)-butan-1-one, 1-(4-fluorophenyl)-4-[4-hydroxy-4-[3-(trifluoromethyl)phenyl]-1-piperidyl]-butan-1-one, 1-[1-[4-(4-fluorophenyl)-4-oxo-butyl]-4-piperidyl]-3H-benzoimidazol-2-one, 1-[1-[4-(4-fluorophenyl)-4-oxo-butyl]-3,6-dihydro-2H-pyridin-4-yl]-3H-benzoimidazol-2-one, 8-[4-(4-fluorophenyl)-4-oxo-butyl]-1-phenyl-1,3,8-triazaspiro[4,5]decan-4-one, 1-[4,4-bis(4-fluorophenyl)butyl]-4-[4-chloro-3-(trifluoromethyl)phenyl]-piperidin-4-ol, derivatives, variants or combinations thereof.

As used herein, the term “ischemia” refers to a reduction or cessation of blood flow to the a cell or tissue in a patient that may be global or focal. For example, global cerebral ischemia refers to reduction of blood flow within the cerebral vasculature resulting from systemic circulatory failure caused by, e.g., shock, cardiac failure, or cardiac arrest. Ischemia leads to a “shock” that is the state in which failure of the circulatory system to maintain adequate cellular perfusion results in reduction of oxygen and nutrients to tissues. Ischemia may be found anywhere in the body, e.g., heart, brain, circulation, etc. In some cases, as taught herein, the ischemic site will be the site of surgery where ischemia occurs, whether planned or not. Within minutes of circulatory failure, blockage or surgical shunting, tissues become ischemic, particularly in the heart and brain. The most common form of shock is cardiogenic shock, e.g., from severe depression of cardiac performance. Cardiogenic shock is often the result of a myocardial infarction. Cardiac pump failure may also result from acute myocarditis, depression of myocardial contractility following a cardiac arrest or prolonged cardiopulmonary bypass. Mechanical abnormalities, such as severe valvular stenosis, massive aortic or mitral regurgitation, acutely acquired ventricular septal defects, can also cause cardiogenic shock by reducing cardiac output. Additional causes of cardiogenic shock include cardiac arrhythmia, such as ventricular fibrillation.

As used herein, the terms “patient” or “subject” refer to animals, e.g., mammals, including but not limited to humans, pigs, cats, dogs, rodents, or cattle including but not limited to, sheep, goats and cows. Most often, patients are humans. The compositions and method of the present invention may be adapted for the treatment of ischemic brain injury, such as a stroke or those injuries associated with, and secondary to, traumatic brain damage.

As used herein, “sigma-1 receptor antagonists” refers to compounds that are antagonists of opioid sigma-1 receptors, e.g., human opioid sigma-1 receptors. The sigma-1 receptor antagonists have also been found to protect cells or tissues from injury caused by ischemia, e.g., cardiac or brain injury.

The present invention includes compounds having the general Formula I:

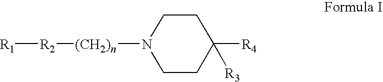

For example, one such compound includes Formula I in which R₁ is a substituted phenyl (e.g., 4-chlorophenyl, 4-bromophenyl or 4-fluorophenyl). Although the most common position for the substitution is the 4 position the phenyl may be substituted at other positions as well. Generally, R₁ may be a chlorophenyl, a bromophenyl, a fluorophenyl, a trichloromethane, a tribromomethane, a trifluoromethane, a dichloromethane, a dibromomethane, a difluoromethane, a chloromethane, a bromomethane or a fluoromethane. In addition, the phenyl may be di-substituted with individually a hydroxy, an alkoxy, an alkoxyalkyl, a hydroxyl, a hydroxyalkyl, an alkenyl, an amino, a nitrate, an alkylamino, a dialkylamino, a nitro, an aryl, an alkylaryl, an arylalkoxy, a cycloalkyl, a carboxyl, a carbonyl, a halogen, a haloalkyl, a haloalkoxy, a heteroayl, a heterocyclic ring, an arylheterocyclic ring, an amido, an alkylamido, a carboxylic ester or a carboxylic acid. In some embodiments, R1 may be a bis-substituted phenyl bonded to R2, e.g., 4,4-bis(4-fluorophenyl)butyl.

The R₂ is generally a ketone (e.g., —CO—) but may also be reduced to a hydroxyl group (e.g., —COH—). The (CH2)n alkyl group includes 0, 1, 2, 3, 4, 5 or 6 carbons, corresponding to an n equal to 0 to 6 carbons, however, the three carbon alkyl is most common. Other embodiments may have a (CH2)n alkyl group having one or more double bonds form an alkenyl and/or substitutions including a hydroxy, an alkoxy, an alkoxyalkyl, a hydroxyl, a hydroxyalkyl, an alkenyl, an amino, a nitrate, an alkylamino, a dialkylamino, a nitro, an aryl, an alkylaryl, an arylalkoxy, a cycloalkyl, a carboxyl, a carbonyl, a halogen, a haloalkyl, a haloalkoxy, a heteroayl, a heterocyclic ring, an arylheterocyclic ring, an amido, an alkylamido, a carboxylic ester or a carboxylic acid. The combination of R2 and the alkyl group generally include a lower alkyl group having a total of four carbons and a ketone group.

R₃ may be a hydroxyl, a hydrogen, a lone pair of electrons or electrons involve in the bonds of the ring. In addition, R₃ may be a hydroxy, an alkoxy, an alkoxyalkyl, a hydroxyl, a hydroxyalkyl, an alkenyl, an amino, a nitrate, an alkylamino, a dialkylamino, a nitro, an aryl, an alkylaryl, an arylalkoxy, a cycloalkyl, a carboxyl, a carbonyl, a halogen, a haloalkyl, a haloalkoxy, a heteroayl, a heterocyclic ring, an arylheterocyclic ring, an amido, an alkylamido, a carboxylic ester or a carboxylic acid.

Generally, R₄ is a substituted phenyl (e.g., 4-chlorophenyl, 4-bromophenyl or 4-fluorophenyl) attached to the ring at the one position. Although the most common position for the substitution is the 4 position the phenyl may be substituted at other positions as well. Generally, R₄ may be a chlorophenyl, a bromophenyl, a fluorophenyl, a trichloromethane, a tribromomethane, a trifluoromethane, a dichloromethane, a dibromomethane, a difluoromethane, a chloromethane, a bromomethane or a fluoromethane. In addition, the phenyl of R₄ may be di-substituted with individually a hydroxy, an alkoxy, an alkoxyalkyl, a hydroxyl, a hydroxyalkyl, an alkenyl, an amino, a nitrate, an alkylamino, a dialkylamino, a nitro, an aryl, an alkylaryl, an arylalkoxy, a cycloalkyl, a carboxyl, a carbonyl, a halogen, a haloalkyl, a haloalkoxy, a heteroayl, a heterocyclic ring, an arylheterocyclic ring, an amido, an alkylamido, a carboxylic ester or a carboxylic acid. One di-substituted phenyl includes 4-chloro-3-(trifluoromethyl)phenyl.

The R₁, R₂, R₃, and R₄ groups of Formula I may be substituted with one or more groups including a hydroxyl group, an alkoxy group, an alkoxyalkyl group, a hydroxyl group, a hydroxyalkyl group, an alkenyl group, an amino group, a nitrate group, an alkylamino group, a dialkylamino group, a nitro group, an aryl group, an alkylaryl group, an arylalkoxy group, a cycloalkyl group, a carboxyl group, a carbonyl group, a halogen group, a haloalkyl group, a haloalkoxy group, a heteroayl group, a heterocyclic ring, an arylheterocyclic ring, an amido group, an alkylamido group, a carboxylic ester, a carboxylic acid and a combinations thereof.

In addition, the substituted groups themselves may be substituted with a hydroxy, an alkoxy, an alkoxyalkyl, a hydroxyl, a hydroxyalkyl, an alkenyl, an amino, a nitrate, an alkylamino, a dialkylamino, a nitro, an aryl, an alkylaryl, an arylalkoxy, a cycloalkyl, a carboxyl, a carbonyl, a halogen, a haloalkyl, a haloalkoxy, a heteroayl, a heterocyclic ring, an arylheterocyclic ring, an amido, an alkylamido, a carboxylic ester, a carboxylic acid and combinations thereof.

Specific embodiments of the compound of Formula I include, e.g., 4-[4-(4-chlorophenyl)-4-hydroxy-1-piperidyl]-1-(4-fluorophenyl)-butan-1-one, 1-(4-chlorophenyl)-4-[4-(4-chlorophenyl)-4-hydroxy-1-piperidyl]-butan-1-one, 4-[4-(4-bromophenyl)-4-hydroxy-1-piperidyl]-1-(4-fluorophenyl)-butan-1-one, 1-(4-fluorophenyl)-4-[4-hydroxy-4-[3-(trifluoromethyl)phenyl]-1-piperidyl]-butan-1-one, 1-[1-[4-(4-fluorophenyl)-4-oxo-butyl]-4-piperidyl]-3H-benzoimidazol-2-one, 1-[1-[4-(4-fluorophenyl)-4-oxo-butyl]-3,6-dihydro-2H-pyridin-4-yl]-3H-benzoimidazol-2-one, 8-[4-(4-fluorophenyl)-4-oxo-butyl]-1-phenyl-1,3,8-triazaspiro[4,5]decan-4-one and 1-[4,4-bis(4-fluorophenyl)butyl]-4-[4-chloro-3-(trifluoromethyl)phenyl]-piperidin-4-ol, wherein the compound is provided in an amount sufficient to protect cells, tissues and patients from an ischemic event, e.g., a stroke.

The term “lower alkyl” as used herein refers to branched or straight chain alkyl groups having one to ten carbon atoms, including methyl, ethyl, propyl, isopropyl, n-butyl, t-butyl, neopentyl and the like.

The term “alkoxy” as used herein refers to RO— wherein R is a lower alkyl group as defined herein. “Alkoxy groups” include, for example, methoxy, ethoxy, t-butoxy and the like.

The term “alkoxyalkyl” as used herein refers to an alkoxy group as previously defined appended to an alkyl group as previously defined. Examples of alkoxyalkyl include, but are not limited to, methoxymethyl, methoxyethyl, isopropoxymethyl and the like.

The term “hydroxy” as used herein refers to —OH.

The term “hydroxyalkyl” as used herein refers to a hydroxy group as previously defined appended to a lower alkyl group as previously defined.

The term “alkenyl” as used herein refers to a branched or straight chain C2-C20 hydrocarbon which also comprises one or more carbon-carbon double bonds.

The term “amino” as used herein refers to —NH2.

The term “nitrate” as used herein refers to —O—NO2.

The term “alkylamino” as used herein refers to RNH— wherein R is as defined in the specification. Alkylamino groups include, for example, methylamino, ethylamino, butylamino, and the like.

The term “dialkylamino” as used herein refers to RR*N— wherein R and R* re independently selected from lower alkyl groups as defined herein. Dialkylamino groups include, for example dimethylamino, diethylamino, methyl propylamino and the like.

The term “nitro” as used herein refers to the group —NO2 and “nitrosated” refers to compounds that have been substituted therewith.

The term “nitroso” as used herein refers to the group —NO and “nitrosylated” refers to compounds that have been substituted therewith.

The term “aryl” as used herein refers to a mono- or bi-cyclic carbocyclic ring system having one or two rings including, but not limited to, phenyl, naphthyl, tetrahydronaphthyl, indanyl, indenyl, tetralyl, benzoimidyl, piperidyl and the like. Aryl groups (including bicyclic aryl groups) can be unsubstituted or substituted with one, two or three substituents independently selected from lower alkyl, haloalkyl, alkoxy, amino, alkylamino, dialkylamino, hydroxy, halo, and nitro.

The term “alkylaryl” as used herein refers to a lower alkyl radical to which is appended an aryl group. Arylalkyl groups include, for example, benzyl, phenylethyl, hydroxybenzyl, fluorobenzyl, fluorophenylethyl and the like.

The term “arylalkoxy” as used herein refers to an alkoxy radical to which is appended an aryl group. Arylalkoxy groups include, for example, benzyloxy, phenylethoxy, chlorophenylethoxy and the like.

The term “cycloalkyl” as used herein refers to an alicyclic group comprising from about 3 to about 7 carbon atoms including, but not limited to, cyclopropyl, cyclobutyl, cyclopentyl, cyclohexyl and the like.

The term “bridged cycloalkyl” as used herein refers to two or more cycloalkyl radicals fused via adjacent or non-adjacent carbon atoms, including, but not limited to, adamantyl and decahydronapthyl.

The term “cycloalkoxy” as used herein refers to RO— wherein R is cycloalkyl as defined in this specification. Representative examples of alkoxy groups include cyclopropoxy, cyclopentyloxy, and cyclohexyloxy and the like.

The term “arylthio” as used herein refers to RS— wherein R is an aryl group as defined herein.

The term “alkylsulfinyl” as used herein refers to R—S(O)2- wherein R is as defined in this specification.

The term “caboxamido” as used herein refers to —C(O)NH2.

The term “carbamoyl” as used herein refers to —O—C(O)NH2.

The term “carboxyl” as used herein refers to —CO2H.

The term “carbonyl” as used herein refers to —C(O)—.

The term “halogen” or “halo” as used herein refers to I, Br, Cl, or F.

The term “haloalkyl” as used herein refers to a lower alkyl radical to which is appended one or more halogens. Representative examples of haloalkyl group include trigluoromethyl, chloromethyl, 2-bromobutyl, 1-bromo-2-chloro-pentyl and the like.

The term “haloalkoxy” as used herein refers to a haloalkyl radical as defined herein to which is appended an alkoxy group as defined herein. Representative examples of haloalkoxy groups include 1,1,1-trichloroethoxy, 2-bromobutoxy and the like.

The term “heterocyclic ring” as used herein refers to any 3-, 4-, 5-, 6-, 7-, 8-, 9-, 10-, 11-, 12-, 13-, 14-, 15- or 16-membered nonaromatic ring containing at least one nitrogen atom, oxygen atom, or sulfur atom which is bonded to an atom which is not part of the heterocyclic ring.

The term “arylheterocyclic ring” as used herein refers to a bi- or tri-cyclic ring comprised of an aryl ring as previously defined appended via two adjacent carbon atoms of the aryl group to a heterocyclic ring as previously defined.

The term “heterocyclic compounds” as used herein refers to mono- and poly-cyclic compounds containing at least one heteroaryl or heterocyclic ring, as defined herein.

The term “amido” as used herein refers to —NH—C(O)—R wherein R is a lower alkyl, aryl, or hereroaryl group, as defined herein.

The term “alkylamido” as used herein refers to RN—C(O)—R* wherein R and R* are individually a lower alkyl, aryl, or hereroaryl group, as defined herein.

The term “carboxylic ester” as used herein refers to —C(O)OR, wherein R is a lower alkyl group as defined herein.

The term “carboxylic acid” as used herein refers to —C(O)OH.

Certain compositions of the present invention have been used at much higher doses as anti-psychotics, however, the present invention includes compositions and methods for protection of cells, tissues and patients against the effects of ischemic trauma at non-therapeutic doses of the agents and compounds taught herein. The compounds of the present invention may be provided in low-dosage forms that are adapted for the delivery of lower-doses of the compounds to a patient in need of protection from ischemia.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions incorporated herein by reference.

For example, the butyrophenones and/or Sigma-1 receptor antagonists may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The butyrophenones and/or Sigma-1 receptor antagonists may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidyicholines, mixtures thereof, and the like.

In another example, the butyrophenones and/or Sigma-1 receptor antagonists may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the butyrophenones and Sigma-1 receptor antagonists may be coupled one or more biodegradable polymers to achieve controlled release of the butyrophenones or Sigma-1 receptor antagonists, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the butyrophenones and/or Sigma-1 receptor antagonists and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs and alveoli, the butyrophenones and/or Sigma-1 receptor antagonists may also be delivered as an intranasal form via use of a suitable intranasal vehicle. For dermal and transdermal delivery, the butyrophenones and/or Sigma-1 receptor antagonists may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of butyrophenones and/or Sigma-1 receptor antagonists may include the following forms.

Capsules. Capsules may be prepared by filling standard two-piece hard gelatin capsules each with 1.0 to 50.0 milligrams of powdered butyrophenones and/or Sigma-1 receptor antagonists, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active butyrophenones and/or Sigma-1 receptor antagonists are prepared and injected by using a positive displacement pump into gelatin to form soft gelatin capsules containing, e.g., 10-50 milligrams of the active ingredient. The capsules are washed and dried.

Tablets. A large number of tablets are prepared by conventional procedures so that the dosage unit was 0.5-5.0 milligrams of butyrophenones and/or Sigma-1 receptor antagonists per kilogram weight, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption. For example, for a patient that is 80 kg, a dosage form with 80 mg would dose at 1.0 mg/kg. For pediatric patients, the dosage often be reduced to half of the adult dosage, e.g., 0.5 mg/kg.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 10.0 mg of finely divided butyrophenones and/or Sigma-1 receptor antagonists, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of butyrophenones and/or Sigma-1 receptor antagonists. Such kits may filter include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Pharmaceutically acceptable carrier. A carrier can be a solid or liquid and the type is generally chosen based on the type of administration being used. The butyrophenones and/or Sigma-1 receptor antagonists can be coadministered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

The present inventors recognized a critical nexus between possible neuroprotective role for antipsychotic drugs and oxidative stress. For example, at high concentrations (30-200 μM) the benzazepine atypical antipsychotics (e.g., clozapine, olanzapine and quetiapine) partially protect PC12 cells from toxic insults like hydrogen peroxide, MPP+ and Aβ25-35 (Wei et al., 2003a; Wei et al., 2003b; Li et al., 2003). Older studies report that the typical antipsychotic haloperidol is neuroprotective against PCP/ketamine-induced neuronal injury, which is an effect mediated by heat shock proteins (Sharp et al., 1992; Nakki et al., 1996).

An ischemic event sets in motion a cascade that leads ultimately to an overproduction of NO. Briefly, excessive glutamate overactivates NMDA receptors leading to an abnormal rise in intracellular calcium which in turn intensely activates calcium-sensitive nNOS and results in the overproduction of NO (Iadecola, et al., 1997). Ischemia induced by tMCAO results in rapid and large elevations in the level of extracellular glutamate (˜25-fold within 20-30 minutes) in the ischemic penumbral cortex (Takagi, et al., 1993). Cortical levels of NO are elevated up to ˜150-fold in ischemic tissue during a tMCAO-induced ischemic cerebral stroke and again become elevated as much as ˜50-fold during reperfusion (Zhang, et al., 1995).

Treatments or conditions that limit NO overproduction in neurons protect the brain from ischemia. For instance, selective inhibitors of nNOS reduce ischemic stroke volume (Zhang, et al., 1996; Yoshida et al., 1994; Willmot et al., 2005). In addition, less severe neurological deficits and smaller ischemic lesion volumes are observed in homozygous nNOS knockout mice compared to their wild type littermates following a tMCAO stroke (Hara, et al., 1996). Moreover, nNOS-selective inhibitors do not provide any further neuroprotection against tMCAO-induced stroke in homozygous nNOS knockout mice (Goyagi, et al., 2001). Together these studies indicate that a significant portion of the damage from an ischemic cerebral stroke is due to an nNOS-mediated overproduction of NO and that preventing this reduces the amount of brain damage caused by the ischemic event.

Sigma-1 ligands protect against chemical ischemia-induced and glutamate-induced toxicity in rat cortical cultures (DeCoster, et al., 1995; Nishikawa, et al., 2000; Kume, et al., 2002). However, both sigma-1-selective agonists like (+)-SKF 10,047 and Sigma-1 antagonists like haloperidol were shown to be protective and both have potencies around 1-4 μM. The necessity for such extremely high concentrations (micromolar) and the finding that both an agonist and an antagonist produced the same effect is consistent with a direct channel blocking effect for both (+)-SKF 10,047 and haloperidol on NMDA receptors (Nishikawa, et al., 2000; Kume, et al., 2002). Thus, while it is true that haloperidol can antagonize both sigma-1 receptors and NMDA receptors, it is essential to recognize that haloperidol's affinity is three orders of magnitude lower for NMDA receptors than for sigma-1 receptors (Fletcher et al., 1995; Coughenour and Cordon, 1997; Whittemore, et al., 1997; Gallagher, et al., 1998; Shim et al., 1999; Hayashi, et al., 1999; Bowen, et al., 1990; Ganaphthy, et al., 1999; Nishikawa, et al., 2000). Consequently, the inventors' recognized that the concentration or dose of haloperidol used in each study is a critical factor when considering its potential receptor targets. This does not mean that sigma-1 receptors cannot mediate excitotoxicity evoked by NMDA receptor stimulation, as this has been demonstrated (Bhardwaj et al., 1998), rather only that the protective effect of low nanomolar concentrations haloperidol cannot be due to a direct blockade of NMDA receptors.

There is good evidence that sigma-1 receptors are capable of mediating protection against cerebral ischemic stroke. For example, the high affinity sigma-1 selective ligand 4-phenyl-1-(4-phenylbutyl)piperidine (PPBP) decreases transient focal ischemia-induced brain injury in rats, cats and mice (Takahashi, et al. 1995; Takahashi, et al. 1996; Goyagi, et al., 2001). There is also evidence that sigma-1 receptors protect by attenuating nNOS-mediated production of NO. For instance, ischemic lesion volume following a tMCAO stroke is greatly reduced in homozygous nNOS knockout mice compared to their wild type littermates (Goyagi, et al., 2001), and neither the Sigma-1 ligand PPBP nor selective nNOS inhibitors provide further protection to the nNOS knockout mice. The present inventors recognized that protection by butyrophenone antipsychotics like haloperidol is due to antagonism of the Sigma-1 receptor.

The present inventors determined that low doses of butyrophenones are neuroprotective against traumatic brain injuries, some of which are prevalent in aging populations, through the reduction of secondary oxidative stress-related damage. Neuroprotection was shown using a variety of in vitro and in vivo techniques. In vitro molecular mechanisms responsible for the neuroprotection against oxidative stress-induced cell death may be determined in a glutamate-induced oxidative stress (OS) hippocampal HT-22 cell model. The glutamate-induced oxidative stress model using the immortalized mouse hippocampal neuron cell line HT-22 is our in vitro protection screening assay, because HT-22 cells lack NMDA receptors (Zaulyanov, et al., 1999; Ishige, et al., 2001) and compounds that are protective in this in vitro assay are protective in rats in vivo (see for example, Prokai, et al. 2003). In vivo neuroprotection against ischemic brain stroke may be measured using a well-established transient middle cerebral artery occlusion (MCAO) model to induce an ischemic stroke in rats (Longa, et al. 1989). The degree of in vivo neuroprotection may be determined using a reliable measure of stroke severity: differential triphenyltetrazolium staining to histologically assess infarct volume (Dettners, et al., 1994; Yang, et al., 1998).

The present inventors also found that the butyrophenone (commonly used as antipsychotics) have specific structural features that allow then to serve as neuroprotectants against ischemic injury. Due to their long history of usage, approval and known dosages and side-effects, the compounds have an accelerated potential for use in cerebral stroke patients. The drugs in preclinical studies of ischemic stroke have are already used in humans to treat other symptom modalities (i.e., psychosis, agitation/aggression and deviant sexual behavior) and their safety profiles at the doses disclosed herein are well within the safety margins and well-established. For example, the acute dose to be protective against tMCAO in the rat studies (i.e., 0.05 mg/kg) disclosed herein is a dose that results in ˜65-70% occupancy of rat D2 dopamine receptors. This level of D2 dopamine receptor blockade produces behaviors in rats indicative of an antipsychotic effect in humans, but not behaviors indicative of extrapyramidal or neuroendocrine side-effects (Wadenberg et al., 2000). In humans, this same level of chronic D2 receptor occupancy produces an antipsychotic effect in schizophrenics without a risk of extrapyramidal or neuroendocrine side-effects (Kapur et al., 2000; Wadenberg et al., 2000). As taught herein, one important application is to limit the damage induced by cerebral ischemic stroke in the acute and subacute phases (up to 2 days after stroke), even the small risk of side-effects associated with long term chronic inactivation of D2 dopamine receptors are not a concern. Further, unlike some atypical antipsychotics, haloperidol, does not significantly increase blood glucose levels (Dwyer et al., 2003; Newcomer et al., 2002), which is an undesirable effect during a cerebral ischemic stroke (Kawai et al., 1997; Farrokhnia et al., 2005; Paolino and Gamer, 2005). Brain stoke is the third leading cause of death and the leading cause of disability in the U.S.A. (Rosenberg et al., 1996; Mancia, 2004). An estimated 700,000 strokes occur every year and about 29% of these may be recurrent strokes (Radziszewska et al, 2005; AHA, 2005). Incidents of first-ever major stroke approximately double every decade of life over the age of 55 to about 17% in those aged 85 and older (Rothwell et al., 2004). The U.S. census bureau projects that the elderly U.S. population (>65 years of age) will increase from a current 13% of the population to 20% of the population by the year 2050 (website reference 1). Thus, the total number of brain stroke victims is projected to rise dramatically in the coming decades, as the population that is most at risk continues to grow (e.g., over 1.1 million strokes occurring annually by 2025, Broderick, 2004). Over 80% of strokes are classified as ischemic strokes meaning that they are due to a deficiency in blood flow leading to oxygen and nutrient deprivation and a state of oxidative stress (Mohr et al., 1978; Elkind, 2003; Manica, 2004). Even relatively short durations of oxidative stress can trigger cell death (Tan et al., 1998). Approximately 94% of those presenting with ischemic stroke are 45 years of age or older (Grau et al., 2001), and youthful homeostatic systems that are generally effective in combating oxidative injury are compromised in aging populations (Droge, 2003; Junqueira et al., 2004). Although there is still debate as to the exact pattern and time course of neurological deficits following ischemic stroke due to coronary artery bypass grafting (CABG) (Baskett et al., 2005), other types of surgery (Rothwell et al., 1996; Wong et al., 2000; Kawaharada et al., 2005) or non-surgical etiologies, vascular dementia is now believed to be the most common form of dementia in the elderly (Roman, 2002). The high rates of disabling ischemic stroke in the elderly combined with an increase in the percentage of the population who are elderly, makes cerebral ischemic stroke one of the nation's most urgent health care concerns.

General treatment strategies for ischemic stroke include prevention, limiting the damage caused by an ongoing stroke and post-stroke rehabilitation. Prevention strategies rely on reducing the underlying risk factors for stroke. Some risk factors can be addressed by behavioral modifications, such as cessation of tobacco smoking, increasing one's regular physical activity and healthy diet (Goldstein et al., 2001; Broderick, 2004). Others risk factors, such as hypertension, diabetes mellitus, atrial fibrillation, left ventricular hypertrophy by EKG, and clinical coronary disease, can be reduced by pharmacotherapeutic management of the underlying disease state (Goldstein et al., 2001; Radziszewska et al, 2005). However, some primary risk factors for stroke, such as age, sex or ethnicity, are not modifiable. In addition, various types of surgical procedures that may be needed pose a considerable risk (2-11%) for ischemic cerebral stroke (Rothwell et al., 1996; Wong et al., 2000; Baskett et al., 2005; Kawaharada et al., 2005). Consequently, there is still a need for new and improved medications that either prevent or limit the extent of the damage induced by an ischemic cerebral stroke.

A typical acute therapy approach is to stop an ongoing ischemic stroke as it is occurring by rapidly dissolving the blood clot responsible for the vascular occlusion. The only currently approved thrombolytic or “clot-busting” agent for the treatment of acute stroke in the U.S.A. is intravenously administered tissue plasminogen activator (tPA) (Manica, 2004). Placebo controlled clinical studies with intravenous tPA have shown a 12% increase in the number of stroke victims that recover normal neurological function three months after the stroke (NINDS rt-PA Stroke Group, 1995; Alberts, 1997). However, the intravenous tPA approach is used infrequently (<10% of acute ischemic stroke patients, Kleindorfer et al., 2004) (Magid et al, 2005), since strokes are often not identified within a suitable time frame (Schwamm et al., 2005); intravenous tPA works best when administered within 90 minutes after ischemic stroke and by three hours the benefit diminishes while the risk for a thrombolytic stroke increases (Hacke et al., 2004). The risk for thrombolytic stroke is due to tPA converting plasminogen, a blood clotting factor, to plasmin, a blood clot dissolving proteolytic enzyme (Grandjean et al., 2004). In addition, tPA can apparently activate excitotoxic NMDA receptors, which in turn may exacerbate oxidative stress-induced cell death (Nicole et al., 2001; Traynelis and Lipton, 2001).

The two most popular classes of approved clinical pharmacotherapies for the prevention of recurrent stroke are blood thinners and statins. Blood thinners are classified as antiplatelet agents or anticoagulants. The rationale for the use of blood thinners is that they prevent the formation of blood clots, thereby preventing the occurrence of future strokes. However, blood thinners have considerable drawbacks. For instance, the orally-active anticoagulant warfarin has a narrow therapeutic window, which necessitates continuous monitoring of its levels and dietary restrictions (e.g., avoidance of foods and supplements rich in vitamin K). Antiplatelet agents, such as dipyridamole, clopidogrel and aspirin, carry a risk for gastrointestinal bleeding and hemorrhagic stroke (Radziszewska et al., 2005). The protection by statins (e.g., pravstatin, atorvastatin, lovastatin and simvastatin) is complex as they have a multitude of effects (Laufs, 2003). With respect to the current proposal, the most relevant effect is an increase in the levels of vascular NO via upregulation of endothelial NOS (eNOS), which has been shown to protect against ischemic stroke in mice (Huang et al., 1996; Endres et al., 1998; Laufs et al., 2000; Laufs et al., 2002). The mechanisms of protection appear to be due to an antithrombic effect as well as an anti-inflammatory effect associated with improved endothelial function, and consequently, enhanced vascularization (Laufs et al., 2000; Laufs et al., 2003). Despite their beneficial effects, there are safety concerns over the HMG CoA reductase inhibiting activity of statins. Clinical studies have revealed that statins increase the risk of potentially life-threatening myopathies due to reductions in Coenzyme Q10 whose production dependent upon HMG CoA reductase activity (Pasternak et al., 2002). Preclinical studies suggest that termination of statin treatment results in thrombus formation and a loss of protection (Gertz et al., 2003). Promising preclinical pharmacotherapies for combating oxidative stress related to ischemic cerebral stroke are non-feminizing estrogens (Liu et al., 2002), other antioxidants (Bhavnani, 2003; Calabrese et al., 2003; Granot and Kohen, 2004), NMDA receptor antagonists (Farber et al., 2002; Petty et al., 2003; Li et al., 2004), and the Sigma-1 receptor ligand 4-phenyl-1-(4-phenylbutyl)piperidine (Takahashi et al., 1995; Takahashi et al., 1996; Goyagi et al., 2001; Goyagi et al. 2003).

There is evidence that sigma-1 receptors are capable of mediating protection against cerebral ischemic stroke. For example, the high affinity sigma-1 selective ligand 4-phenyl-1-(4-phenylbutyl)piperidine (PPBP) decreases transient focal ischemia-induced brain injury in rats, cats and mice (Takahashi et al. 1995; Takahashi et al. 1996; Goyagi et al., 2001). There is also evidence that sigma-1 receptors protect by attenuating nNOS-mediated production of NO. For instance, ischemic lesion volume following a tMCAO stroke is greatly reduced in homozygous nNOS knockout mice compared to their wild type littermates (Goyagi et al., 2001), and neither the Sigma-1 ligand PPBP nor selective nNOS inhibitors provide further protection to the nNOS knockout mice. The dose-effect, time course and mechanism of sigma-1 receptor-mediated protection by butyrophenone antipsychotics in response to oxidative stress-related cell death in vitro and ischemic cerebral stroke in vivo are disclosed. These results support the proposition that protection by butyrophenone antipsychotics like haloperidol is due to antagonism of the Sigma-1 receptor.

This application builds on the inventors' recognition that ischemic damage due to a transient middle cerebral artery occlusion is reduced 50% following coincident application of a low dose (0.05 mg/kg) of the antipsychotic haloperidol, a commonly used antipsychotic. This protection against oxidative stress-related cell death is not due to excitotoxic receptor blockade, because the protective dose in vivo and the protective potency in vitro for haloperidol are 2-3 orders of magnitude lower than what is needed to block NMDA receptors.

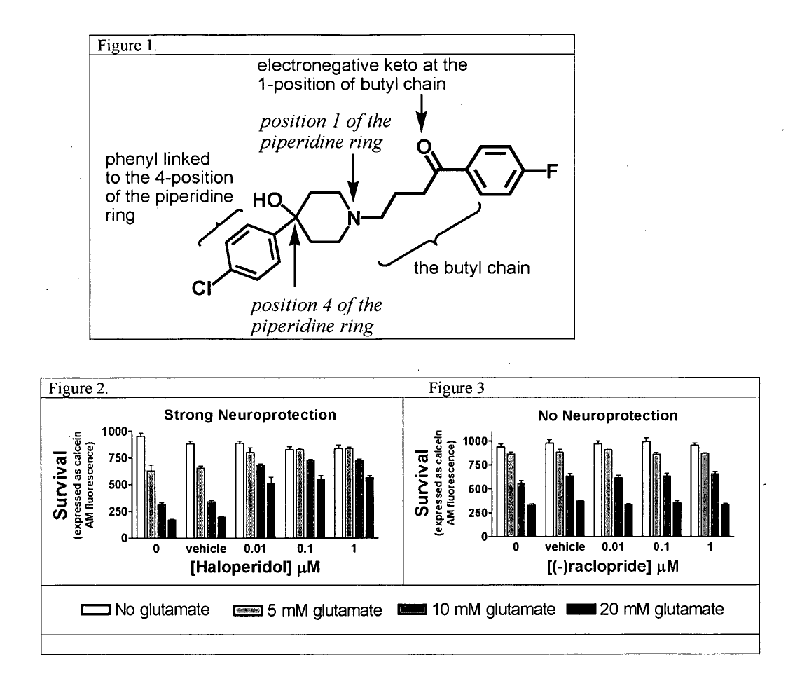

It was found that butyrophenone drugs with specific structural features protect against oxidative stress-related cell death by antagonizing Sigma-1 receptors. Haloperidol is the prototypical example of an antipsychotic drug possessing these specific structural features (i.e., a butyrophenone core structure, and a 1-linked phenyl and an electronegative moiety along the butyl chain as substructural features, FIG. 1).

FIG. 1 shows the chemical structure of haloperidol highlighting core and substructural features. The butyrophenone core structure on the right hand side is shown as thicker lines. The substructural features include a phenyl ring connected to the 4-position of the piperidine ring (left hand side) and the electronegative keto moiety at position 4 along the butyl chain.

FIGS. 2 and 3 are graphs that summarizes example of some raw data for the in vitro protection assay using the glutamate-induced, oxidative stress-related HT-22 cell model. FIG. 2 is a graph that shows an example of some raw data for the in vitro protection assay in a glutamate-induced, oxidative stress-related HT-22 cell model. The glutamate-induced oxidative stress model using the immortalized mouse hippocampal neuron cell line HT-22 is our in vitro protection screening assay, because HT-22 cells lack NMDA receptors (Zaulyanov et al., 1999; Ishige et al., 2001) and compounds that are protective in this in vitro assay are protective in rats in vivo (see for example, Prokai et al., 2003).

FIG. 3 is a graph that shows raw data for the in vitro protection assay in the glutamate-induced, oxidative stress-related HT-22 cell model with S-(−)-raclopride as an example of an antipsychotic drug that provides no neuroprotection. Increasing concentrations of glutamate result in higher levels of oxidative stress leading to higher levels of cell death. Cell survival is measured with the fluorescent vital dye Calcein AM. Haloperidol is and example of an antipsychotic drug that provides strong neuroprotection and S-(−)-raclopride is an example of an antipsychotic drug that provides no neuroprotection. In vivo protection is infarct volume (extent of the ischemic lesion) measured 24 hrs after reperfusion.

Examples of derivatives for the compounds of the present invention include metabolically-stable bioisoster equivalent to an electronegative moiety at position 4 along the butyl chain of haloperidol in an effort to retain high affinity Sigma-1 receptor antagonism while drastically reducing interactions with D2-like (i.e., D2, D3 and D4) dopamine receptors.

The present inventors have found that antipsychotic drugs might be protective under conditions of oxidative stress began by screening them in an in vitro protection assay. A glutamate-induced oxidative stress model was used with the immortalized mouse hippocampal neuronal cell line HT-22 as an in vitro protection screening assay, because HT-22 cells lack NMDA receptors (Zaulyanov et al., 1999; Ishige et al., 2001) and compounds that are protective in this in vitro assay are protective in rats in vivo (see for example, Prokai et al., 2003). In the HT-22 cell model, extracellular application of glutamate induces oxidative stress by reversing the glutamate/cystine-antiporter (Li et al., 1998; Ishige et al., 2001). This depletes HT-22 cells of the intracellular cystine needed for production of the endogenous antioxidant glutathione. This in turn leads to an increase in reactive oxygen species, which is followed by elevations in intracellular calcium and cell death (Ishige et al., 2001). A screen of neuroprotective effects for antipsychotic drugs was restricted to only those whose safety profile and clinical efficacy for the treatment of schizophrenia have been well-characterized, and included antipsychotics representative of a broad range of chemical/structural classes: phenothiozine, thioxanthine, benzodiazepine, benzazoline, substituted benzamide and butyrophenone (Table 1). Remarkably, only the butyrophenone haloperidol demonstrated a potent and efficacious protective effect.

Since haloperidol is known to block several molecular targets with low nanomolar affinity (e.g., certain dopamine, serotonin and sigma receptor subtypes), we initiated a second round of screening designed to investigate the receptor profile responsible for haloperidol's robust protective effect (Table 2). Included in this second screen are compounds with a range of selectivities for the different subfamilies or subtypes of dopamine, serotonin and sigma receptors. Remarkably, the only two compounds that mimicked the protective potency of haloperidol are high affinity selective antagonists of the Sigma-1 or the D4 dopamine receptor (i.e., BD1063 and L741,742, respectively, Table 2). However, only one of the two high affinity D4-selective antagonists (i.e., L741,742) has a potent effect similar to haloperidol. The other D4-selective antagonist L745,870 and the D2-like antagonist pimozide provide very weak or no protection (Table 2). Yet pimozide, L745,870, L741,742 and haloperidol all have high affinities for the cloned D4 receptor (Table 3). This lack of a consistent correlation for these four ligands with respect to their affinity for the cloned D4 dopamine receptor and their in vitro protective potencies suggests that the potent protective effect observed for haloperidol is independent of the D4 dopamine receptor.

Since the high-affinity Sigma-1-selective antagonist BD1063 emulates haloperidol's potent protective effect, the alternative explanation is that the in vitro protection by haloperidol is due to blockade of the Sigma-1 receptor. To investigate this possibility further, we developed an assay for reliably measuring the affinity of ligands for the Sigma-1 receptor. Since [³H]-(+)-pentazocine is the only readily-available radioligand suitable for a Sigma-1 binding assay and it binds other receptors (e.g. opioid receptors) as well, we sought a cell line devoid of [³H]-(+)-pentazocine specific binding to serve as a null background for the expression of a cloned Sigma-1 receptor. It was found that untransfected MCF-7 cells have no detectable specific binding for the Sigma-1 receptor radioligand [³H]-(+)-pentazocine (FIG. 4).

FIG. 4 is a graph that shows the [³H]-(+)-pentazocine saturation isotherm binding to a clonal human MCF-7 cell line stably expressing the human Sigma-1 receptor. The average affinity (K_{D}) and Bₘₐₓ values (n=3) are: 347 nM and 108 pmoles/mg protein. No specific [³H]-(+)-pentazocine binding was detected in untransfected MCF-7 cells, indicating the absence of endogenous Sigma-1 or opioid receptors. Untransfected MCF-7 cells have no detectable specific binding for the Sigma-1 radioligand [3H]-(+)-pentazocine. MCF-7 cells lack full length, pharmacologically-active Sigma-1 receptors (Vilner et al., 1995; Seth et al., 1998; Yamamoto et al., 1999; Shamsul et al., 2002; also see FIG. 3); the exon 3 splice variant is missing the portion of the receptor that binds Sigma-1 ligands (Ganaphthy et al., 1999).

Table 1 shows the neuroprotection screening with antipsychotic drugs in the in vitro glutamate-induced oxidative stress HT-22 cell model. All antipsychotic drugs screened here have been approved for clinical use in the treatment of other disorders (e.g., psychosis in schizophrenia). Potency and efficacy values represent protection against oxidative stress induced by application of 20 mM glutamate. See also FIGS. 2 and 3 for examples of some raw data.

TABLE 1

| | | Potency | | |
|---|---|---|---|---|
| Chemical/Structural | | (EC₅₀ ± SEM, | Efficacy ± | |
| Class of Drug | Drug Name | nM) | SEM | Comments |
| Butyrophenone | Haloperidol | 1.2 ± 0.4 | 65 ± 2.5 | Typical |
| | | | | antipsychotic |
| Phenothiozine | Chlorpromazine | >1000 | 0 | Typical |
| | | | | antipsychotic. |
| | Thioridazine | >1000 | 0 | Typical |
| | | | | antipsychotic. |
| | Perphenazine | >1000 | 0 | Typical |
| | | | | antipsychotic. |
| | Flupenazine | >1000 | 0 | Typical |
| | | | | antipsychotic. |
| Thioxanthene | Chlorprothixene | >1000 | 0 | Typical |
| | | | | antipsychotic. |
| Substituted | S-(−)-Raclopride | >1000 | 0 | Typical |
| Benzamide | | | | antipsychotic. |
| | | | | D2/D3 selective |
| | | | | antagonist |
| Dibenzoazepine | Clozapine | >1000 | 0 | Atypical |
| | | | | antipsychotic. |
| | Loxapine | >1000 | 0 | (Probably) a |
| | | | | typical |
| | | | | antipsychotic. |
| Benzazoline | Risperidone | >1000 | 0 | Atypical |
| | | | | antipsychotic. |

Table 2 shows the identification of receptor targets mediating protection: focus on dopamine, serotonin and sigma receptors. Protective activity was determined in vitro using the glutamate-induced oxidative stress HT-22 cell model.

TABLE 2

| | Potency | | |
|---|---|---|---|
| | (EC₅₀ ± | Efficacy ± | |
| Compound | SEM, nM) | SEM | Comments |
| Pimozide | >1000 | 0 | D2-like receptor antagonist. |
| L741626 | >1000 | 0 | D2-selective antagonist |
| Raclopride | >1000 | 0 | D2/D3-selective antagonists |
| L745870 | 895 ± 736 | 50 ± 9.6 | D4-selective antagonist |
| L741742 | 1.2 ± 0.82 | 61 ± 2.7 | D4-selective antagonist |
| PD168077 | >1000 | 0 | D4-selective agonist |
| Ketanserin | >1000 | 0 | 5HT-like receptor antagonist |
| Amoxapine | >1000 | 0 | 5HT2-like antagonist |
| Mirtazapine | >1000 | 0 | 5HT2-like antagonist |
| BD1063 | 5.9 ± 3.5 | 61 ± 7.5 | Sigma-1-selective antagonist |
| PRE-084 | >1000 | 0 | Sigma-1-selective agonist |
| SM-21 | >1000 | 0 | Sigma-2-selective antagonist |

Note that D2-like includes D2, D3 and D4 subtypes and 5HT-like includes 5HT1, 5HT2, 5HT6 and 5HT7 subtypes.

Table 3 shows the lack of correspondence between in vitro protective activity and affinity for the cloned D4 dopamine receptor.

TABLE 3

| | Affinity for | | |
|---|---|---|---|
| | the cloned D4 | | |
| Drug/ | dopamine | In vitro Protective | |
| Compound | receptor | Potency | |
| Name | (Kᵢ, nM) | (EC₅₀ ± SEM, nM) | Comments |
| Pimozide | 1.8^{a} | >1000 | D2-like antagonist |
| Haloperidol | 2.3^{b} | 1.2 ± 0.4 | D2-like antagonists |
| L745,870 | 0.44^{c} | 895 ± 736 | D4-selective |
| | | | antagonists |
| L741,742 | 3.5^{d} | 1.2 ± 0.82 | D4-selective |
| | | | antagonists |

^{a}Burstein et al., 2005
^{b}Seeman P, Van Tol, 1994
^{c}Kulagowski et al., 1996
^{d}Rowley et al., 1996.

Table 4 shows a correspondence between in vitro protective activity and affinity for the cloned sigma-1 receptor: focus on structure-activity and structure-affinity relationships for butyrophenones, Sigma-1-selective antagonist and two compounds with high affinity for the D4 dopamine receptor. Most of these butyrophenones are approved for clinical use in the treatment of schizophrenia (e.g., United States, Europe and Asia) or deviant sexual behavior (Europe).

TABLE 4

| | Affinity for the cloned | Potency | |
|---|---|---|---|
| | Sigma-1 receptor | (EC₅₀ ± SEM nM) | |
| Drug/Compound Name | (Kᵢ ± SEM, nM) | & Efficacy ± SEM | Comments |
| Haloperidol | 1.7 ± 0.46 | 1.2 ± 0.4 | Typical antipsychotic. |
| | | 65 ± 2.5 | Butyrophenone |
| | | | structure. |
| Trifluperidol | 3.3 ± 0.06 | 7.5 ± 6.3 | Haloperidol congener. |
| | | 55 ± 3.0 | |
| Chlorohaloperidol | 1.5 ± 0.36 | 4.3 ± 1.7 | Haloperidol congener. |
| | | 64 ± 3.4 | |
| Bromperidol | 1.2 ± 0.21 | 0.95 ± 0.82 | Haloperidol congener. |
| | | 67 ± 9.8 | |
| Penfluridol | 53 ± 15 | 350 ± 246 | Pimozide-like. A |
| | | 80 ± 20 | diphenylbutylpiperidine |
| Haloperidol metabolite II | 1.5 ± 0.47 | 12.9 ± 12.2 | A reduced keto. Similar |
| (Reduced haloperidol) | | 58 ± 5.6 | affinity as haloperidol |
| | | | for Sigma-1, but ~3200- |
| | | | fold less affinity for D2 |
| | | | dopamine receptors. |
| Spiperone | 1054 ± 334 | 2737 ± 714 | Typical antipsychotic. |
| | | 100 | Butyrophenone |
| | | | structure. |
| Droperidol | 2240 ± 499 | 5271 ± 2230 | Spiperone-like |
| | | 100 | |
| Benperidol | 252 ± 42 | 1157 ± 498 | Spiperone-like |
| | | 66 ± 23 | |
| L745870 | 63 ± 13 | 895 ± 736 | D4-selective antagonist. |
| | | 50 ± 9.6 | Non-butyrophenone |
| L741742 | 4.8 ± 0.41 | 1.2 ± 0.82 | D4-selective antagonist. |
| | | 61 ± 2.7 | Non-butyrophenone. |
| BD1063 | 3.1 ± 1.4 | 5.9 ± 3.5 | Sigma-1-selective |
| | | 61 ± 7.5 | antagonist. |
| | | | Non-butyrophenone. |

These findings are consistent with the report that MCF-7 cells lack full length, pharmacologically-active Sigma-1 receptors (Vilner et al., 1995; Seth et al., 1998; Yamamoto et al., 1999; Shamsul et al., 2002; also see FIG. 3); the exon 3 splice variant is missing the portion of the receptor that binds Sigma-1 ligands (Ganaphthy et al., 1999). Stable expression of the full-length, cloned Sigma-1 receptor in MCF-7 cells results in high affinity [³H]-(+)-pentazocine binding within the range expected for a Sigma-1 receptor (Seth et al., 1998; Mei and Pasternak, 2001). Using this assay system, we were able to demonstrate that the Sigma-1-selective antagonist BD1063 and the ligand L741,742 bind to cloned Sigma-1 receptors with affinities similar to their protective potencies in HT-22 cells (Table 4). The discovery that L741,742, which is a ligand touted as a “D4-selective” antagonist (Rowley et al., 1996), is protective and binds with high affinity to the Sigma-1 receptor prompted the investigation of the relationship between in vitro protective potency and affinity for the cloned Sigma-1 receptor.

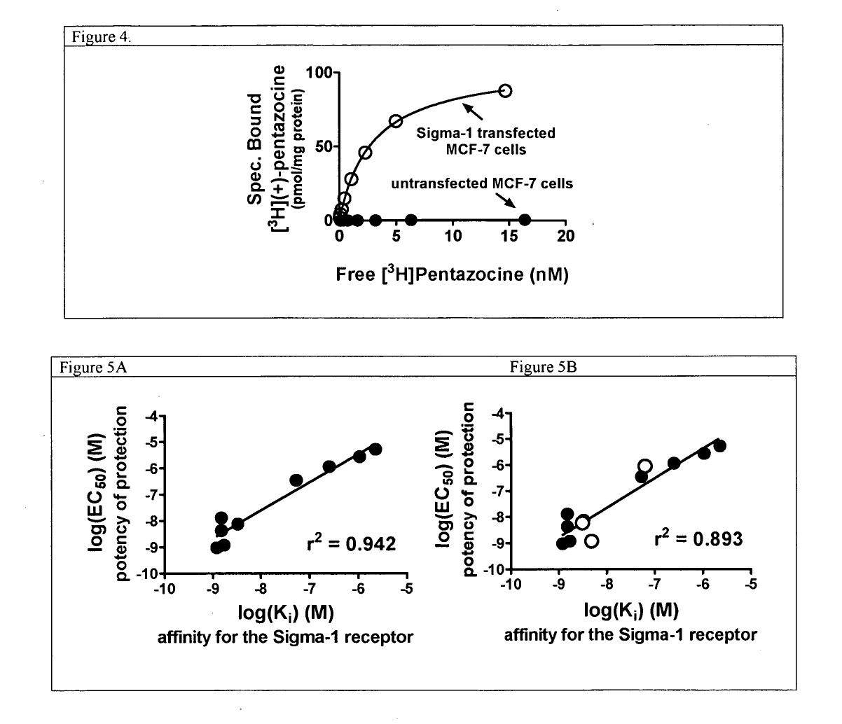

FIGS. 5A and 5B are graphs that show an analysis of the potency of in vitro protection and affinity for the cloned Sigma-1 receptor. Solid circles represent butyrophenone antipsychotics and open circles represent non-butyrophenone structures. FIG. 5A shows a correlation for butyrophenone antipsychotics only. FIG. 5B shows a correlation of butyrophenone antipsychotics plus the non-butyrophenone compounds BD1063, L741,742 and L745,870.

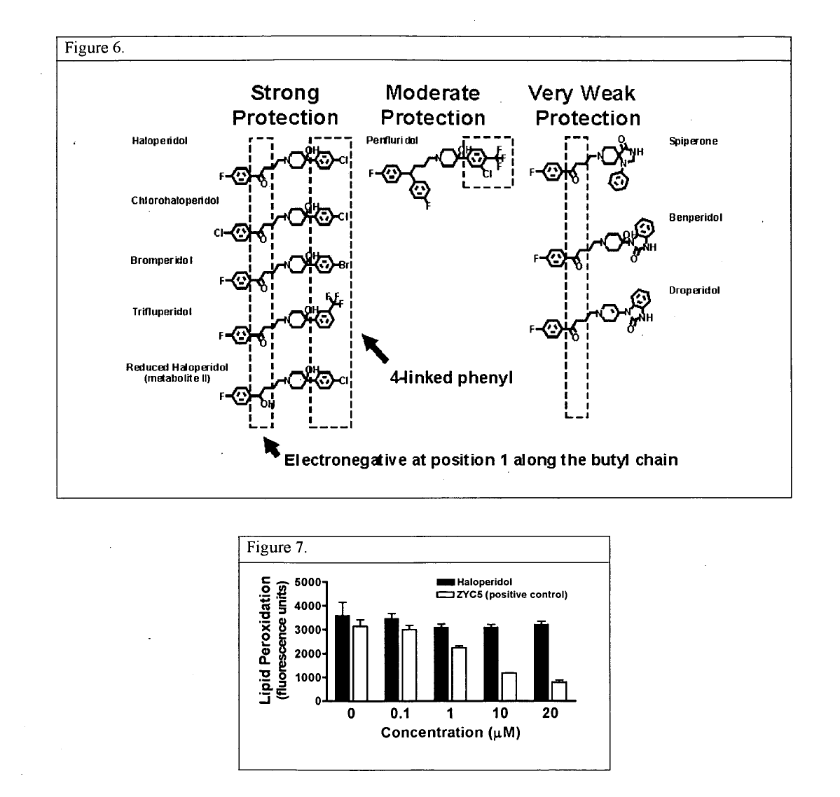

A striking positive correlation exists between the in vitro protection afforded by the nine (9) antipsychotic drugs belonging to the butyrophenone structural class and their affinities for the Sigma-1 receptor (r²=0.942, Table 4 and FIG. 5A). In other words, the most potent butyrophenones are those with the highest affinity for the Sigma-1 receptor. In addition, there are clear substructural requirements for Sigma-1 receptor-mediated protection by butyrophenones: potent protection and high affinity binding to the Sigma-1 receptor require the presence of both a 1-linked phenyl and an electronegative moiety at position 4 along the butyl chain (FIG. 6).

FIG. 6 shows the structure-protection relationships of butyrophenones in the in vitro HT-22 cell model of oxidative stress in accordance with the present invention. Strong protection is defined as EC₅₀<20 nM, moderate protection as 20 nM>EC₅₀<1000 nM, and very weak protection as EC₅₀>1000 nM, respectively. Substructural features important for the protective effect are boxed with dashed lines. The presence of a 1-linked phenyl is more critical for activity than having an electronegative moiety at position 4 along the butyl chain. A high potency effect requires the presence of both substructural features.

For example, the butyrophenones spiperone, benperidol and droperidol (right side of FIG. 6) each have an electronegative keto moiety (C═O) along the butyl chain, but they do not have a 1-linked phenyl substructure and they do not provide potent protection nor do they have high affinity for the sigma-1 receptor. Penfluridol (middle of FIG. 6) has a 1-linked phenyl substructure, but lacks an electronegative moiety along the butyl chain and it has moderate protective potency and moderate affinity for the Sigma-1 receptor. Haloperidol, chlorohaloperidol, bromperidol, trifluperidol and reduced haloperidol (left side of FIG. 6) each have a 1-linked phenyl substructure in combination with an electronegative moiety along the butyl chain (either a keto (CO) or a hydroxyl (—OH)) and all provide high potency protection and high affinity for the Sigma-1 receptor. Although reduced haloperidol (also known as metabolite II) is strongly protective, no protection was observed for either of the other two metabolites of haloperidol (also known as metabolites I and III), which are formed by a dissection of the core structure of haloperidol (data not shown). Together these structure-activity relationships indicate that the specific butyrophenone substructural features required for strong protection against glutamate-induced oxidative stress in HT-22 cells are a combination of a 1 linked phenyl and an electronegative moiety along the butyl chain (Table 4 and FIGS. 5 and 6). Because protective potency is highly correlated with affinity for the Sigma-1 receptor, these structural features are important for Sigma-1 receptor structure-affinity relationships as well.

Remarkably, a strong correlation was found between protective potency and affinity for the cloned Sigma-1 receptor observed for the butyrophenones, which persists even when other compounds with different structures (i.e., non-butyrophenone compounds) are added to the data set (r²=0.893, Table 4 and FIG. 5, right panel). In contrast, a potent sigma-2 receptor selective antagonist SM-21 has no protective effect (Table 5). These structure-activity and structure-affinity results provide further evidence that it is the Sigma-1 receptor that is the critical molecular target mediating protection in an in vitro assay. Moreover, it is specifically antagonism of Sigma-1 receptors that is important for protection, because haloperidol and BD1063 are high affinity Sigma-1 receptor antagonists and their protective effect is not mimicked by the high affinity sigma-1-selective agonist PRE-084 (Table 5).

FIG. 7 is a graph that shows that Haloperidol is not an antioxidant. Antioxidant activity was measured by the ability of compounds to prevent ferric chloride-induced lipid peroxidation in rat brain membranes. The antioxidant ZYC5 is a known antioxidant control. Figure shows that the protection by halperidol cannot be attributed to an antioxidant effect, as has been demonstrated for some non-feminizing estrogens (Liu et al., 2002 and see data for ZYCS in FIG. 7), because even very high concentrations of haloperidol failed to prevent ferric chloride-induced peroxidation of brain lipids in vitro. In summary, in vitro protection against glutamate-induced oxidative stress in HT-22 cells is mediated by antagonism of Sigma-1 receptors.

Table 5 shows that a sigma-1 agonist and a sigma-2 antagonist fail to protect in the in vitro glutamate-induced oxidative stress HT-22 cell model.

TABLE 5

| | Affinity for | | |
|---|---|---|---|
| | the Sigma-1 | Potency | |
| | receptor | (EC₅₀ ± SEM, nM) & | |
| Compound | (Kᵢ, nM) | Efficacy ± SEM | Comments |
| BD1063 | 3.1 | 5.9 ± 3.5 | Sigma-1-selective |
| | | 61 ± 7.5 | antagonist. |
| | | | Non-butyrophenone. |
| PRE-084 | 2.2^{a} | >1000 | Sigma-1-selective |
| | | | agonist |
| SM-21 | UD | >1000 | Sigma-2-selective |
| | | | antagonist |

^{a}Su et al., 1991.
UD undetermined in receptor assays.

The in vitro data suggest that protection against oxidative stress-related cell death is mediated via Sigma-1 receptor antagonism, whether a butyrophenone antipsychotic possessing the critical substructural features (FIGS. 1 & 6) could produce a similar protection in vivo was investigated. A rat tMCAO model of ischemic cerebral stroke was selected for this purpose, because it is a well-established in vivo model of oxidative stress with good face value. Ovariectomized (OVX) female rats were selected for studies to approximate the estrogen depleted state in post-menopausal (elderly) women and to eliminate the protective effects of endogenous cycling estrogens, which reduce infarct volume.

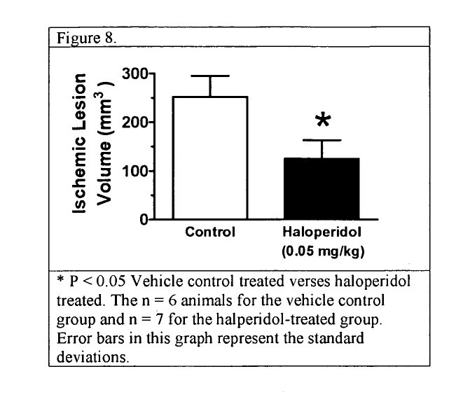

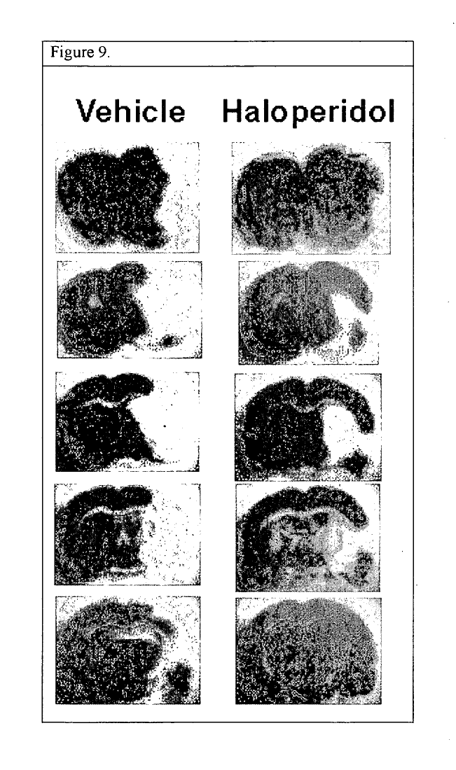

FIG. 8 is a graph that shows the in vivo protection against tMCAO induced brain injury in ovariectomized female Sprague-Dawley rats assessed as infarct volume. The images in FIG. 9 shows that Haloperidol protects against tMCAO brain injury. Representative triphenyltetrazolium-stained coronal brain slices (top to bottom corresponds to anterior to posterior) from tMCAO (hemi)stroked ovariectomized female Sprague-Dawley rats and protection by an acute low dose of haloperidol (0.05 mg/kg). Dead (non-respiring) tissue appears white while living tissue appears red (or dark gray in gray scale). The right hand side of each brain is the stroked side.

An acute low dose of haloperidol (0.05 mg/kg) administered to ovariectomized female Sprague-Dawley rats immediately following the induction of a transient MCA occlusion (60 minute duration) provided a 50% reduction in infarct volume when assessed 24 hrs after the stroke (FIGS. 8 and 9). This low dose of haloperidol was chosen, because it is a dose that produces behaviors in rats indicative of antipsychotic action in humans, but not those indicative of extrapyramidal side-effects: a dose that results in an in vivo D2 dopamine receptor occupancy of about 65-70%. Importantly, this low dose is at least 500 times lower than that required to elicit any effect on NMDA receptors, which have very low affinity for haloperidol (>1 μM). Although an in vitro screening assay indicated no protective role for dopamine receptors. The rationale for this test dose relates to the possible repurposing of certain butyrophenone antipsychotics as protectants to treat ischemic cerebral stroke with no risk of extrapyramidal side-effects. At a 0.05 mg/kg dose in OVX rats, haloperidol produced a significant and large reduction in ischemic lesion volume, thus validating the approach and demonstrating a robust in vivo protective effect for this butyrophenone antipsychotic (FIGS. 8 and 9).

Tertiary Data Mining. The present inventors next sought to confirm their results in vivo. Using in silico data mining, the present inventors searched one such database for the present invention in patient populations that had never been analyzed for a tertiary effect, that is, a known effect of the treatment or a side-effect. The tertiary effect in the present invention is the long-term outcome of a heretofore unknown effect of the compounds described herein. This novel method of analysis was used to extract stroke data that was not gathered or correlated, until now, with the use of FDA approved anti-psychotics, such as Haloperidol. Table 6 summarizes a statistically significant sample of patient data from just one state, Kentucky, as related to occlusions (stenosis, precerebral arteries and cerebral arteries) as well as transient cerebral ischemia. A clear correlation and reduction in the potential and effect of ischemia was found to be correlated with use of Haloperidol.

To estimate the neuroprotective effects of Haloperidol, a retrospective study was conducted that estimates whether Haloperidol use impacts the probability for stroke. To estimate the effect of Haloperidol on stroke outcomes, an administrative data set constructed by the Kentucky Department for Medicaid Services that contains all Medicaid claims and detailed eligibility data for calendar year 1997 was used as a source of data for data mining. To allow for appropriate treatment time periods, the data was restricted to Medicaid members who had at least 11 months of eligibility during the year. In addition, since the primary outcome measure was stroke, the data was limited to members who were aged 41 or greater during the year. With these limitations, there were 145,576 members in the 1997 data set.

In operation, the data was mined as follows. The Medicaid eligibility files contain demographic information for each member, including gender, age, race, and program eligibility type (to document residence in a long term care facility, and Medicare/Medicaid dual eligibility). The claims data files include data on every service payment made on behalf of Medicaid members. Claim data have three general components: a professional component that includes services billed by physicians and other caregivers, a facility component that includes services billed by hospitals and clinics, and a pharmacy component that includes outpatient pharmaceuticals billed by individual pharmacies. All claim files include an identifier to match patients with their eligibility files. Linking patients from their eligibility file with associated claim files allow for the creation of a patient specific data set that includes patient demographics and indicators to identify diagnosis codes and procedure codes. These codes allow for classification of diagnosed diseases and specific medical procedures and specific pharmaceutical treatments for each member in the data set. Using previous work documenting stroke risk factors as a guide (Grau et al. 2001), the following factors were included in the final data set for each member: age, gender, race, long term care residence, dual eligibility, days of eligibility, aspirin therapy, hyperlipidemia diagnosis, hyptertension diagnosis, stroke diagnosis (including date and type), coronary artery bypass graft procedure, coumadin therapy, statin (HMG Co-Reductase inhibitor therapy), haloperidol therapy, initiation date for haloperidol therapy, and the length of haloperidol therapy. Each factor was used to estimate a logistic regression predicting stroke during 1997.

Table 6 presents descriptive statistics for each factor used in the model. The table lists the minimum, maximum, mean, and standard deviation for each factor in the data set. Since most of the factors are binary variables, the mean indicates the percentage of cases where the factor score is 1. So, for example, the stroke factor has a mean of 0.045, which indicates about 4.5% of members in the data set had a stroke during 1997. The diagnosis factors for hyperlipidemia and hypertension indicate whether each member had one of the reported diagnosis codes on any claim during 1996 or 1997. The factors for haloperidol use are split into several categories, indicating the duration of therapy ranging from a single prescription during the year, to 180 days or greater therapy duration. In addition, since time sequence of therapy and stroke date is an important criteria, the haloperidol factors were only coded ‘1’ where the patient initiated haloperidol therapy during 1997 and did not have a stroke during the year, or, where the patient initiated haloperidol therapy during 1997 before the first reported stroke date. More specifically, members were not coded who began haloperidol treatment after reporting a stroke during 1997 as a valid therapy window-those patients are coded ‘0’ since they were not on haloperidol therapy at the time of their first stroke.

### Kentucky Medicaid Program, Haloperidol Stroke Model, 1997 Calendar Year

Age restrictions: >40
Eligibility Restrictions=11 months or greater during 1997
Haloperidol days supplied >=180 days during 1997
Date of Stroke restricted after initiation of Haloperidol therapy
Type of Stroke restricted to ICD-9 codes:
433 Occlusion and stenosis of precerebral arteries
434 Occlusion of cerebral arteries
435 Transient cerebral Ischemia

TABLE 6
Descriptive Statistics N = 145,576

| | | | | Std. |
|---|---|---|---|---|
| Variables | Minimum | Maximum | Mean | Deviation |
| Age | 41 | 109 | 64.043 | 14.937 |
| Male | 0 | 1 | 0.349 | 0.477 |
| White | 0 | 1 | 0.826 | 0.379 |
| Long Term Care | 0 | 1 | 0.146 | 0.353 |
| Medicare/Medicaid Dual | 0 | 1 | 0.767 | 0.423 |
| Days of Eligibility in 1997 | 1 | 364 | 316.446 | 96.796 |
| >30 day Aspirin Therapy | 0 | 1 | 0.124 | 0.330 |
| Hyperlipidemia Diagnosis in 1996 or 1997 | 0 | 1 | 0.136 | 0.343 |
| Hypertension Diagnosis in 1996 or 1997 | 0 | 1 | 0.389 | 0.488 |
| Stroke type = 433, 434, 435 | 0 | 1 | 0.045 | 0.207 |
| Coronary Artery Bypass Graft 36.xx | 0 | 1 | 0.061 | 0.240 |
| >30 day Coumadin Therapy in 1997 | 0 | 1 | 0.048 | 0.213 |
| >30 day Statin Therapy in 1997 | 0 | 1 | 0.017 | 0.129 |
| Any Haloperidol Prescription in 1997 | 0 | 1 | 0.027 | 0.163 |
| 60 days or Greater Haloperidol in 1997 | 0 | 1 | 0.018 | 0.133 |
| 90 days or Greater Haloperidol in 1997 | 0 | 1 | 0.015 | 0.123 |
| 120 days or Greater Haloperidol in 1997 | 0 | 1 | 0.014 | 0.116 |
| 150 days or Greater Haloperidol in 1997 | 0 | 1 | 0.012 | 0.110 |
| 180 days or Greater Haloperidol in 1997 | 0 | 1 | 0.011 | 0.105 |

Next, the inventors sought to further investigate and cull from the database disaggregated data for a Haloperidol Stroke Model (Table 7) and a Haloperidol Stroke Model controlling for coronary artery bypass graft (CABG) (Table 8). Table 7 presents the reports the logistic regression predicting stroke during 1997. The model uses 145,576 patients where 6,773 had at least one stroke diagnosis. All factors except race are significant at the 0.001 level. In addition to the regression coefficients, the table reports the Odds Ratio for each factor in the model. The Odds Ratios simplify interpretation and indicate the relative impact of each factor on the probability of each patient having a stroke during 1997.

Of course, age is an important predictor for the probability of having a stroke. Since the age variable is coded in unit years, the Odds Ratio indicates that for each year older than 40, the probability for stroke increases by about 3.1%. Other demographics indicate that males are about 11% more likely to stroke than females, long term care facility patients are about 58% more likely to stroke. Controls for therapeutic factors indicate that patients on Coumadin therapy are about 2.7 times more likely to have a stroke during the year, and patients on statin drugs are about 1.7 times more likely to have a stroke during the year.

Those patients with a reported history for hyperlipidemia are about 1.5 times more likely to have a stroke, and those patients with a reported history of hypertension are about 2.4 times more likely to have a stroke. Looking at those patients who had heart surgery, patients who have a coronary artery bypass graft procedure are about 2.7 times more likely to have a stroke. Finally, the haloperidol factor indicates that patients who had at least 180 days of haloperidol therapy were about 38% less likely to have a stroke, thus providing supporting evidence that haloperidol may provide neuroprotective benefits to patients.

TABLE 7
Haloperidol Stroke Model N = 145,576

| Logistic Regression | | | | | | | 95.0% C.I. for | |
|---|---|---|---|---|---|---|---|---|
| Results for Stroke | | | | | | Odds | EXP(B) | |
| Variable | B | S.E. | Wald | df | Sig. | Ratio | Lower | Upper |
| Age | 0.03 | 0.001 | 810.318 | 1 | 0.000 | 1.031 | 1.029 | 1.033 |
| Male | 0.104 | 0.028 | 13.506 | 1 | 0.000 | 1.110 | 1.050 | 1.174 |
| White | 0.021 | 0.034 | 0.378 | 1 | 0.539 | 1.021 | 0.955 | 1.091 |
| Long Term Care | 0.46 | 0.036 | 164.421 | 1 | 0.000 | 1.584 | 1.476 | 1.699 |
| Hyperlipidemia Diagnosis | 0.438 | 0.033 | 175.859 | 1 | 0.000 | 1.549 | 1.452 | 1.653 |
| in 1996 or 1997 | | | | | | | | |
| Hypertension Diagnosis in | 0.889 | 0.028 | 1016.367 | 1 | 0.000 | 2.432 | 2.303 | 2.569 |
| 1996 or 1997 | | | | | | | | |
| >30 day Coumadin | 1.018 | 0.038 | 729.76 | 1 | 0.000 | 2.769 | 2.572 | 2.981 |
| Therapy in 1997 | | | | | | | | |
| >30 day Statin Therapy in | 0.474 | 0.067 | 49.416 | 1 | 0.000 | 1.606 | 1.408 | 1.833 |
| 1997 | | | | | | | | |
| 180 days or Greater | −0.464 | 0.128 | 13.018 | 1 | 0.000 | 0.629 | 0.489 | 0.809 |
| Haloperidol in 1997 | | | | | | | | |
| Coronary Artery Bypass | 1.028 | 0.039 | 702.609 | 1 | 0.000 | 2.795 | 2.591 | 3.016 |
| Graft 36.xx | | | | | | | | |
| Constant | −5.955 | 0.082 | 5213.527 | 1 | 0.000 | 0.003 | | |

Stroke, 1 = 6.773 0 = 138.803

Table 8 reports the results for a model restricted to those patients who had CABG surgery during 1997. Of the 11,444 patients in the study who had a CABG procedure during 1997, 1,303 had a stroke following the surgery. Results were generally consistent with the full stroke model, but for those patients who were on haloperidol therapy, the risk of stroke decreased by about 60% (although the factor is significant only at the 0.07 level) compared to about a 40% reduction for the general model. Thus, haloperidol appears to have a greater impact on those patients with a higher risk of stroke. Overall, this retrospective data analysis indicates that haloperidol does appear to reduce the probability for stroke among Medicaid patients.

TABLE 8
Haloperidol Stroke Model
Controlling for Patients with
CABG in 1997
N = 11,444

| Logistic Regression | | | | | | | 95.0% C.I. for | |
|---|---|---|---|---|---|---|---|---|
| Results for Stroke | | | | | | Odds | EXP(B) | |
| Variable | B | S.E. | Wald | df | Sig. | Ratio | Lower | Upper |
| Age | 0.045 | 0.003 | 258.947 | 1 | 0.000 | 1.046 | 1.04 | 1.052 |
| Male | 0.162 | 0.065 | 6.242 | 1 | 0.012 | 1.175 | 1.035 | 1.334 |
| White | 0.128 | 0.079 | 2.631 | 1 | 0.105 | 1.137 | 0.974 | 1.327 |
| Long Term Care | −0.195 | 0.110 | 3.111 | 1 | 0.078 | 0.823 | 0.663 | 1.022 |
| Hyperlipidemia Diagnosis | 0.159 | 0.069 | 5.272 | 1 | 0.022 | 1.172 | 1.024 | 1.342 |
| in 1996 or 1997 | | | | | | | | |
| Hypertension Diagnosis in | 0.686 | 0.070 | 97.084 | 1 | 0.000 | 1.986 | 1.732 | 2.276 |
| 1996 or 1997 | | | | | | | | |
| >30 day Coumadin | 0.849 | 0.078 | 118.382 | 1 | 0.000 | 2.337 | 2.006 | 2.723 |
| Therapy in 1997 | | | | | | | | |
| >30 day Statin Therapy in | 0.463 | 0.123 | 14.175 | 1 | 0.000 | 1.589 | 1.248 | 2.021 |
| 1997 | | | | | | | | |
| 180 days or Greater | −0.926 | 0.518 | 3.193 | 1 | 0.074 | 0.396 | 0.144 | 1.094 |
| Haloperidol in 1997 | | | | | | | | |
| Constant | −5.529 | 0.194 | 811.471 | 1 | 0.000 | 0.004 | | |

Stroke, 1 = 1.303 0 = 10.141

Table 9 listed the diseases, conditions and/or surgical procedures that will benefit from the present invention. For example, table 9 lists the risk of ischemic cerebral stroke following various surgical procedures.

TABLE 9
Risk of ischemic cerebral stroke following various surgical procedures.

| | Incident of Ischemic | Odds | | |
|---|---|---|---|---|
| Risk condition | Stroke (%) | Ratio | Comment | Reference |
| Any surgery | 4.1 | 3.3 | Risk within 30 days after | Wong et al., |
| General Surgery | 1.9 | 2.5 | surgery | 2000 |
| Orthopedic | 0.9 | 4.0 | | |
| Coronary artery | 1.9-2.7 | 2.5-3.2 | Under 80 years old | Baskett et al., |
| bypass grafting | | | (urgent-emergency) | 2005 |
| (CABG) | 5.4-6.8 | 3.3-4.1 | Over 80 years old | |
| | | | (urgent-emergency) | |
| Carotid | 3.5 | | For symptomatic | Rothwell et al., |
| endarterectomy | | | stenosis | 1996 |
| | 5.2 | | For asymptomatic | |
| | | | stenosis | |
| Surgery to repair | 2.0-11.0 | | Ascending or arch aorta | Kawaharada et |
| aneurysms | 2.0-8.1 | | Descending or | al., 2005 |
| | | | thoracoabdominal aorta | |

Kawaharada N, Morishita K, Fukada J, Hachiro Y, Fujisawa Y, Saito T, Kurimoto Y, Abe T. Stroke in surgery of the arteriosclerotic descending thoracic aortic aneurysms: influence of cross-clamping technique of the aorta. Eur J Cardiothorac Surg. 2005 Apr; 27(4): 622-5.
Baskett R, Buth K, Ghali W, Norris C, Maas T, Maitland A, Ross D, Forgie R, Hirsch G. Outcomes in octogenarians undergoing coronary artery bypass grafting. CMAJ. 2005 Apr 26; 172(9): 1183-6.
Wong GY, Warner DO, Schroeder DR, Offord KP, Warner MA, Maxson PM, Whisnant JP. Risk of surgery and anesthesia for ischemic stroke. Anesthesiology. 2000 Feb; 92(2): 425-32.
Rothwell PM, Slattery J, Warlow CP. A systematic comparison of the risks of stroke and death due to carotid endarterectomy for symptomatic and asymptomatic stenosis. Stroke. 1996; 27: 266-269.

### REFERENCES

AbdelMalik P, Husted J, Chow E W, Bassett A S. Childhood head injury and expression of schizophrenia in multiply affected families. Arch Gen Psychiatry. 2003 March; 60(3):231-6.
Alberts M J. Hyperacute stroke therapy with tissue plasminogen activator. Am J Cardiol. 1997; 80(4C):29D-34D; discussion 35D-39D.
American Heart Association (2005), Heart Disease and Stroke Statistics—2005 Update, American Heart Association, http://www.americanheart.org/downloadable/heart/1105390918119HDSStats2005Update.pdf
Baskett R, Buth K, Ghali W, Norris C, Maas T, Maitland A, Ross D, Forgie R, Hirsch G. Outcomes in octogenarians undergoing coronary artery bypass grafting. CMAJ. 2005 Apr. 26; 172(9):1183-6.
Bhardwaj A, Sawada M, London E D, Koehler R C, Traystman R J, Kirsch J R. Potent sigmal-receptor ligand 4-phenyl-1-(4-phenylbutyl)piperidine modulates basal and N-methyl-D-aspartate-evoked nitric oxide production in vivo. Stroke. 1998 November; 29(11):2404-11.
Berger G E, Wood S, McGorry P D. Incipient neurovulnerability and neuroprotection in early psychosis. Psychopharmacol Bull. 2003 Spring; 37(2):79-101.
Bersani G, Taddei I, Manuali G, Ramieri L, Venturi P, Osborn J, Pancheri Severity of obstetric complications and risk of adult schizophrenia in male patients: a case-control study. J Matern Fetal Neonatal Med. 2003 July; 14(1):35-8.
Bhavnani B R. Estrogens and menopause: pharmacology of conjugated equine estrogens and their potential role in the prevention of neurodegenerative diseases such as Alzheimer's. J Steroid Biochem Mol. Biol. 2003 June; 85(2-5):473-82.
Boksa P, El-Khodor B F. Birth insult interacts with stress at adulthood to alter dopaminergic function in animal models: possible implications for schizophrenia and other disorders. Neurosci Biobehav Rev. 2003 January-March; 27(1-2):91-101.
Bowen W D, Moses E L, Tolentino P J, Walker J M. Metabolites of haloperidol display preferential activity at sigma receptors compared to dopamine D-2 receptors. Eur J. Pharmacol. 1990 Feb. 27; 177(3): 111-8.
Broderick J P. William M. Feinberg Lecture: stroke therapy in the year 2025: burden, breakthroughs, and barriers to progress. Stroke. 2004 35:205-211.
Burstein E S, Ma J N, Wong S, Gao Y, Pham E, Knapp A E, Nash N R, Olsson R, Davis R E, Hacksell U, Weiner D M, Brann M R. Intrinsic Efficacy of Antipsychotics at Human D2, D3, and D4 Dopamine Receptors: Identification of the clozapine metabolite N-desmethylclozapine as a D211)₃ partial agonist. J Pharmacol Exp Ther. 2005 Aug. 31; [Epub ahead of print]
Calabrese V, Butterfield D A, Stella A M. Nutritional antioxidants and the heme oxygenase pathway of stress tolerance: novel targets for neuroprotection in Alzheimers disease. Ital J. Biochem. 2003 December; 52(4): 177-81.
Coughenour L L, Cordon J J. Characterization of haloperidol and trifluperidol as subtype-selective N-methyl-D-aspartate (NMDA) receptor antagonists using [3H]TCP and [3H]ifenprodil binding in rat brain membranes. J Pharmacol Exp Ther. 1997 280:584-592.
Das I, Khan N S, Puri B K, Sooranna S R, de Belleroche J, Hirsch S R. Elevated platelet calcium mobilization and nitric oxide synthase activity may reflect abnormalities in schizophrenic brain. Biochem Biophys Res Commun. 1995; 212:375-380.
DeCoster M A, Klette K L, Knight E S, Tortella F C. Sigma receptor-mediated neuroprotection against glutamate toxicity in primary rat neuronal cultures. Brain Res. 1995 Feb. 6; 671(1):45-53.
Dichter M A, Locke R E. Clinical trials in neuroprotection. 23-25 Jan. 2003, Key Biscayne, Fla., USA. Expert Opin Emerg Drugs. 2003 May; 8(1):267-71.
Droge W. Oxidative stress and aging. Adv Exp Med. Biol. 2003; 543:191-200.
Dwyer D S, Donohoe D. Induction of hyperglycemia in mice with atypical antipsychotic drugs that inhibit glucose uptake. Pharmacol Biochem Behav. 2003 May; 75(2):255-60.
Elkind M S. Stroke in the elderly. Mt Sinai J. Med. 2003 January; 70(1):27-37.
Endres M, Laufs U, Huang Z, Nakamura T, Huang P, Moskowitz M A, Liao J K. Stroke protection by 3-hydroxy-3-methylglutaryl (HMG)-CoA reductase inhibitors mediated by endothelial nitric oxide synthase. Proc Natl Acad Sci USA. 1998 Jul. 21; 95(15):8880-8885.
Farber N B, Kim S H, Dikranian K, Jiang X P, Heinkel C. Receptor mechanisms and circuitry underlying NMDA antagonist neurotoxicity. Mol. Psychiatry. 2002; 7(1):32-43.
Farrokhnia N, Bjork E, Lindback J, Terent A. Blood glucose in acute stroke, different therapeutic targets for diabetic and non-diabetic patients? Acta Neurol Scand. 2005 August; 112(2):81-7.
Fletcher E J, Church X, Abdel-Hamid K, MacDonald J F. Blockade by sigma site ligands of N-methyl-D-aspartate-evoked responses in rat and mouse cultured hippocampal pyramidal neurones. Br J Pharmacol. 1995 116:2791-2800.
Forster M J, Dubey A, Dawson K M, Stutts W A, Lal H, Sohal R S. Age-related losses of cognitive function and motor skills in mice are associated with oxidative protein damage in the brain. Proc Natl Acad Sci USA. 1996 May 14; 93(10):4765-9.
Forster M J, Lal H. Neurobehavioral biomarkers of aging: Influence of genotype and dietary restriction. Biomedical and Environmental Sciences 1991; 4: 144-165.
Forster M, Lal H. Cholinergic modulation of aged-like retention deficits in young autoimmune mice. Int J Devl Neuroscience 1990; 8: 679-687.
Ganaphthy M E, Prasad P D, Huang W, Seth P, Leibach F H, Ganaphthy V. Molecular and ligand-binding characterization of the sigma-receptor in the Jurkat human T lymphocyte cell line. J Pharmacol Exp Ther. 1999 April; 289(1):251-60.
Grandjean C, McMullen P C, Newschwander G. Vampire bats yield potent clot buster for ischemic stroke. J Cardiovasc Nurs. 2004 November-December; 19(6):417-20.
Grau A J, Weimar C, Buggle F, Heinrich A, Goertler M, Neumaier S, Glahn J, Brandt T, Hacke W, Diener H C. Risk factors, outcome, and treatment in subtypes of ischemic stroke: the German stroke data bank. Stroke. 2001 November; 32(11):2559-66.
Gallagher M J, Huang H, Lynch D R. Modulation of the N-methyl-D-aspartate receptor by haloperidol: NR2B-specific interactions. J. Neurochem. 1998 70:2120-2128.
Gertz K, Laufs U, Lindauer U, Nickenig G, Bohm M, Dirnagl U, Endres M. Withdrawal of statin treatment abrogates stroke protection in mice. Stroke. 2003 February; 34(2):551-7.
Goldstein L B, Adams R, Becker K, Furberg C D, Gorelick P B, Hademenos G, Hill M, Howard G, Howard V J, Jacobs B, Levine S R, Mosca L, Sacco R L, Sherman D G, Wolf P A, del Zoppo G J. Primary prevention of ischemic stroke: A statement for healthcare professionals from the Stroke Council of the American Heart Association. Circulation. 2001; 103:163-182.
Goyagi T, Goto S, Bhardwaj A, Dawson V L, Hum P D, Kirsch J R. Neuroprotective effect of sigma(1)-receptor ligand 4-phenyl-1-(4-phenylbutyl)piperidine (PPBP) is linked to reduced neuronal nitric oxide production. Stroke. 2001 July; 32(7):1613-20.
Goyagi T, Bhardwaj A, Koehler R C, Traystman R J, Hum P D, Kirsch J R. Potent sigma 1-receptor ligand 4-phenyl-1-(4-phenylbutyl)piperidine provides ischemic neuroprotection without altering dopamine accumulation in vivo in rats. Anesth Analg. 2003 February; 96(2):532-8.
Granot E, Kohen R. Oxidative stress in childhood—in health and disease states. Clin Nutr. 2004 February; 23(1):3-11.
Green P S, Perez E J, Calloway T, Simpkins J W. Estradiol attenuation of β-amyloid-induced toxicity: a comparison of beta-amyloid-induced toxicity: a comparison of MMT and calcein AM assays. J Neurocytol. 2000 29:419-423.
Greenberg S S, Xie J, Spitzer J J, Wang J F, Lancaster J, Grisham M B, Powers D R, Giles T D. Nitro containing L-arginine analogs interfere with assays for nitrate and nitrite. Life Sci. 1995; 57:1949-1961.
Hacke W, Donnan G, Fieschi C, Kaste M, von Kummer R, Broderick J P, Brott T, Frankel M, Grotta J C, Haley E C Jr, Kwiatkowski T, Levine S R, Lewandowski C, Lu M, Lyden P, Marler J R, Patel S, Tilley B C, Albers G, Bluhmki E, Wilhelm M, Hamilton S; ATLANTIS Trials Investigators; ECASS Trials Investigators; NINDS rt-PA Study Group Investigators. Association of outcome with early stroke treatment: pooled analysis of ATLANTIS, ECASS, and NINDS rt-PA stroke trials. Lancet. 2004; 363:768-774.
Hah J M, Roman L J, Martasek P, Silverman R B, Reduced amide bond peptidomimetics. (4S)-N-(4-amino-5-[aminoakyl]aminopentyl)-N′-nitroguanidines, potent and highly selective inhibitors of neuronal nitric oxide synthase. J. Med. Chem. 2001 44; 2667-2670.
Hara H, Huang P L, Panahian N, Fishman M C, Moskowitz M A. Reduced brain edema and infarction volume in mice lacking the neuronal isoform of nitric oxide synthase after transient MCA occlusion. J Cereb Blood Flow Metab. 1996 July; 16(4):605-11.
Hawk T, Zhang Y Q, Rajakumar G, Day A L, Simpkins J W. Testosterone increases and estradiol decreases middle cerebral artery occlusion lesion size in male rats. Brain Res. 1998 Jun. 15; 796(1-2):296-8.
Hayashi T, Su T P, Kagaya A, Nishida A, Shimizu M, Yamawaki S. Neuroleptics with differential affinities at dopamine D2 receptors and sigma receptors affect differently the N-methyl-D-aspartate-induced increase in intracellular calcium concentration: involvement of protein kinase. Synapse. 1999 31:20-28.
Huang Z, Huang P L, Ma J, Meng W, Ayata C, Fishman M C, Moskowitz M A. Enlarged infarcts in endothelial nitric oxide synthase knockout mice are attenuated by nitro-L-arginine. J Cereb Blood Flow Metab. 1996 September; 16(5):981-7.
Iadecola C. Bright and dark sides of nitric oxide in ischemic brain injury. Trends Neurosci. 1997 March; 20(3):132-139.
Ishige K, Schubert D, Sagara Y. Flavonoids protect neuronal cells from oxidative stress by three distinct mechanisms. Free Radic Biol Med. 2001 Feb. 15; 30(4):433-46.
Junqueira V B, Barros S B, Chan S S, Rodrigues L, Giavarotti L, Abud R L, Deucher G P. Aging and oxidative stress. Mol Aspects Med. 2004 February-April; 25(1-2):5-16.
Kawaharada N, Morishita K, Fukada J, Hachiro Y, Fujisawa Y, Saito T, Kurimoto Y, Abe T. Stroke in surgery of the arteriosclerotic descending thoracic aortic aneurysms: influence of cross-clamping technique of the aorta. Eur J Cardiothorac Surg. 2005 April; 27(4):622-5.
Kawai N, Keep R F, Betz A L. Hyperglycemia and the vascular effects of cerebral ischemia. Stroke. 1997 January; 28(1):149-54.
Kapur S, Zipursky R, Jones C, Remington G, Houle S. Relationship between dopamine D(2) occupancy, clinical response, and side effects: a double-blind PET study of first-episode schizophrenia. Am J Psychiatry. 2000 April; 157(4):514-20.
Kikuchi T, Tottori K, Uwahodo Y, Hirose T, Miwa T, Oshiro Y, Morita S. 7-(4-[4-(2,3-Dichlorophenyl)-1-piperazinyl]butyloxy)-3,4-dihydro-2(1H)-quinolinone (OPC-14597), a new putative antipsychotic drug with both presynaptic dopamine autoreceptor agonistic activity and postsynaptic D2 receptor antagonistic activity. J Pharmacol Exp Ther. 1995 274:329-336.
Klamer D, Engel J A, Svensson L. Effects of phencyclidine on acoustic startle and prepulse inhibition in neuronal nitric oxide synthase deficient mice. Eur Neuropsychopharmacol. 2005 October; 15(5):587-90.
Kleindorfer D, Kissela B, Schneider A, Woo D, Khoury J, Miller R, Alwell K, Gebel J, Szaflarski J, Pancioli A, Jauch E, Moomaw C, Shukla R, Broderick J P; Neuroscience Institute. Eligibility for recombinant tissue plasminogen activator in acute ischemic stroke: a population-based study. Stroke. 2004 February; 35(2):e27-29.
Koponen S, Taiminen T, Portin R, Himanen L, Isoniemi H, Heinonen H, Hinkka S, Tenovuo O. Axis I and II psychiatric disorders after traumatic brain injury: a 30-year follow-up study. Am J Psychiatry. 2002 August; 159(8):1315-21.
Kortagere S, Gmeiner P, Weinstein H, Schetz J A. Certain 1,4-disubstituted aromatic piperidines and piperazines with extreme selectivity for the dopamine D4 receptor interact with a common receptor microdomain. Mol. Pharmacol. 2004 66:1491-1499.
Kulagowski J J, Broughton H B, Curtis N R, Mawer I M, Ridgill M P, Baker R, Emms F, Freedman S B, Marwood R, Patel S, Patel S, Ragan C I, Leeson P D. 3-((4-(4-Chlorophenyl)piperazin-1-yl)-methyl)-1H-pyrrolo-2,3-b-pyridine: an antagonist with high affinity and selectivity for the human dopamine D4 receptor. J Med Chem. 1996 May 10; 39(10):1941-2.
Kume T, Nishikawa H, Taguchi R, Hashino A, Katsuki H, Kaneko S, Minami M, Satoh M, Akaike A. Antagonism of NMDA receptors by sigma receptor ligands attenuates chemical ischemia-induced neuronal death in vitro. Eur J Pharmacol. 2002 Nov. 29; 455(2-3):91-100.
Laufs U, Gertz K, Huang P, Nickenig G, Bohm M, Dirnagl U, Endres M. Atorvastatin upregulates type III nitric oxide synthase in thrombocytes, decreases platelet activation, and protects from cerebral ischemia in normocholesterolemic mice. Stroke. 2000 October; 31(10):2442-9.
Laufs U, Gertz K, Dirnagl U, Bohm M, Nickenig G, Endres M. Rosuvastatin, a new HMG-CoA reductase inhibitor, upregulates endothelial nitric oxide synthase and protects from ischemic stroke in mice. Brain Res. 2002 Jun. 28; 942(1-2):23-30.
Laufs U. Beyond lipid-lowering: effects of statins on endothelial nitric oxide. Eur J Clin Pharmacol. 2003 March; 58(11):719-31.
Li J, Henman M C, Doyle K M, Strbian D, Kirby B P, Tatlisumak T, Shaw G G. The pre-ischaemic neuroprotective effect of a novel polyamine antagonist, N1-dansyl-spermine in a permanent focal cerebral ischaemia model in mice. Brain Res. 2004 Dec. 10; 1029(1):84-92.
Li Y, Maher P, Schubert D. Phosphatidylcholine-specific phospholipase C regulates glutamate-induced nerve cell death. Proc Natl Acad Sci USA. 1998 95:7748-7753.
Liu R, Yang S H, Perez E, Yi K D, Wu S S, Eberst K, Prokai L, Prokai-Tatrai K, Cai Z Y, Covey D F, Day A L, Simpkins J W. Neuroprotective effects of a novel non-receptor-binding estrogen analogue: in vitro and in vivo analysis. Stroke. 2002 October; 33(10):2485-91.
Longa E Z, Weinstein P R, Carlson S, Cummins R. Reversible middle cerebral artery occlusion without craniectomy in rats. Stroke. 1989 January; 20(1):84-91.
Magid D, Naviaux N, Wears R L. Stroking the data: re-analysis of the NINDS trial. Ann Emerg Med. 2005 April; 45(4):385-7.
Mancia G. Prevention and treatment of stroke in patients with hypertension. Clin Ther. 2004; 26:631-648.
Markowska A L, Long S M, Johnson C T, Olton D S. Variable-interval probe test as a tool for repeated measurements of spatial memory in the water maze. Behavioral Neuroscience 1993; 107: 627-632.
McCullough, L. D., Alkayed, N. J., Traystman, R. J., Williams, M. J. and Hurn, P. D., Postischemic estrogen reduces hypoperfusion and secondary ischemia after experimental stroke, Stroke 2001 32:796-802.
McMillan D R, Christians E, Forster M, Xiao X, Connell P, Plumier S C, Zuo X, Richardson J, Morgan S, Benjamin I J. Heat shock transcription factor 2 is not essential for embryonic development, fertility, or adult cognitive and psychomotor function in mice. Mol Cell Biol. 2002 22:8005-8014.
Mei J, Pasternak G W. Molecular cloning and pharmacological characterization of the rat signal receptor. Biochem Pharmacol. 2001 Aug. 1; 62(3):349-55.
Metodiewa D, Koska C. Reactive oxygen species and reactive nitrogen species: relevance to cyto(neuro)toxic events and neurologic disorders. An overview. Neurotox Res. 2000 February; 1(3):197-233.
Newcomer J W, Haupt D W, Fucetola R, Melson A K, Schweiger J A, Cooper B P, Selke G. Abnormalities in glucose regulation during antipsychotic treatment of schizophrenia. Arch Gen Psychiatry. 2002 April; 59(4):337-45.
Mohr J P, Caplan L R, Melski J W, Goldstein R J, Duncan G W, Kistler J P, Pessin M S, Bleich H L. The Harvard Cooperative Stroke Registry: a prospective registry. Neurology. 1978; 28:754-762.
Nicole O, Docagne F, Ali C, Margaill I, Carmeliet P, MacKenzie E T, Vivien D, Buisson A. The proteolytic activity of tissue-plasminogen activator enhances NMDA receptor-mediated signaling. Nat. Med. 2001 January; 7(1):59-64.
NINDS rt-PA Stroke Group. Tissue plasminogen activator for acute ischemic stroke. N Engl J. Med. 1995; 333:1581-1587.
Nishikawa H, Hashino A, Kume T, Katsuki H, Kaneko S, Akaike A. Involvement of direct inhibition of NMDA receptors in the effects of sigma-receptor ligands on glutamate neurotoxicity in vitro. Eur J. Pharmacol. 2000 Sep. 15; 404(1-2):41-8.
Nisenbaum I., Rapoport G, Tollefson G., Gould T. (2003) Panel: “Are antipsychotics neuroprotective in Schizophrenia? Findings from Bedside to Benchside.” Winter Conference on Brain Research, Jan. 25, Brechenridge, Colo.
Paolino A S, Garner K M. Effects of hyperglycemia on neurologic outcome in stroke patients. J Neurosci Nurs. 2005 June; 37(3):130-5. Review.
Pasternak R C, Smith S C Jr, Bairey-Merz C N, Grundy S M, Cleeman J I, Lenfant C; American College of Cardiology; American Heart Association; National Heart, Lung and Blood Institute. ACC/AHA/NHLBI clinical advisory on the use and safety of statins. J Am Coll Cardiol. 2002 Aug. 7; 40(3):567-572.
Petty M A, Neumann-Haefelin C, Kalisch J, Sarhan S, Wettstein J G, Juretschke H P. In vivo neuroprotective effects of ACEA 1021 confirmed by magnetic resonance imaging in ischemic stroke. Eur J. Pharmacol. 2003 Aug. 1; 474(1):53-62.
Prabakaran S, Swatton J E, Ryan M M, Huffaker S J, Huang J J, Griffin J L, Wayland M, Freeman T, Dudbridge F, Lilley K S, Karp N A, Hester S, Tkachev D, Mimmack M L, Yolken R H, Webster M J, Torrey E F, Bahn S. Mitochondrial dysfunction in Schizophrenia: evidence for compromised brain metabolism and oxidative stress. Mol. Psychiatry. 2004 Apr. 20 [Epub ahead of print]
Prokai L., Prokai-Tatrai K., Perjesi P., Zharikova A., Perez E., Liu R., Simpkins S., Quinol-based cyclic antioxidant mechanism in estrogen neuroprotection. Proc. National Academy of Sciences USA 100: 11741-11746, 2003.
Radziszewska B, Hart R G, Wolf P A, D'Agostino R B Sr, Cutler J A. Clinical research in primary stroke prevention: needs, opportunities, and challenges. Neuroepidemiology. 2005; 25:91-104.
Ren, M., Senatorov, V. V., Chen, R. W. and Chuang, D. M., Postinsult treatment with lithium reduces brain damage and facilitates neurological recovery in a rat ischemia/reperfusion model, Proc Natl Acad Sci USA, 100 (2003) 6210-5.
Roman G C. Vascular dementia may be the most common form of dementia in the elderly. J Neurol Sci. 2002 Nov. 15; 203-204:7-10. Review.
Roof R L, Schielke G P, Ren X, Hall E D. A comparison of long-term functional outcome after 2 middle cerebral artery occlusion models in rats. Stroke. 2001 32:2648-2657.
Rosenberg, H M, Ventura, S J, Maurer, J D, Heuser, R L and Freedman, M A (1996) Births and Deaths: United States, 1995. Monthly Vital Statistics Report; Vol. 45, No. 3, Suppl. 2.
Rothwell P M, Slattery J, Warlow C P. A systematic comparison of the risks of stroke and death due to carotid endarterectomy for symptomatic and asymptomatic stenosis. Stroke. 1996; 27:266-269.
Rothwell P M, Coull A J, Giles M F, Howard S C, Silver L E, Bull L M, Gutnikov S A, Edwards P, Mant D, Sackley C M, Farmer A, Sandercock P A, Dennis M S, Warlow C P, Bamford J M, Anslow P; Oxford Vascular Study. Change in stroke incidence, mortality, case-fatality, severity, and risk factors in Oxfordshire, UK from 1981 to 2004 (Oxford Vascular Study). Lancet. 2004 Jun. 12; 363(9425):1925-33.
Rowley M, Broughton H B, Collins I, Baker R, Emms F, Marwood R, Patel S, Patel S, Ragan C I, Freedman S B, Leeson P D (1996) 5-(4-Chlorophenyl)-4-methyl-3-(1-(2-phenylethyl)piperidin-4-yl)isoxazole: a potent, selective antagonist at human cloned dopamine D4 receptors. J. Med. Chem. 39 1943-1945.
Samdani A F, Dawson T M, Dawson V L. Nitric oxide synthase in models of focal ischemia. Stroke. 1997 June; 28(6):1283-1288.
Sastry K V, Moudgal R P, Mohan J, Tyagi J S, Rao G S. Spectrophotometric determination of serum nitrite and nitrate by copper-cadmium alloy. Anal Biochem. 2002 Jul. 1; 306(1):79-82.
Schetz J. A. and Anderson (1995), Glycosylation patterns of membrane proteins in the jellyfish Cyanea capillata, Cell Tissue Res., 279:315-321.
Schetz J A, Benjamin P S, Sibley D R. Nonconserved residues in the second transmembrane-spanning domain of the D(4) dopamine receptor are molecular determinants of D(4)-selective pharmacology. Mol. Pharmacol. 2000; 57:144-52.
Schwamm L H, Pancioli A, Acker J E 3rd, Goldstein L B, Zorowitz R D, Shephard T J, Moyer P, Gorman M, Johnston S C, Duncan P W, Gorelick P, Frank J, Stranne S K, Smith R, Federspiel W, Horton K B, Magnis E, Adams R J; American Stroke Association's Task Force on the Development of Stroke Systems. Recommendations for the establishment of stroke systems of care: recommendations from the American Stroke Association's Task Force on the Development of Stroke Systems. Circulation. 2005; 111:1078-1091.
Seeman P, Van Tol H H. Dopamine receptor pharmacology. Trends Pharmacol Sci. 1994 15:264-70.
Seth P, Fei Y J, Li H W, Huang W, Leibach F H, Ganaphthy V. Cloning and functional characterization of a sigma receptor from rat brain. J Neurochem. 1998 70:922-931.
Shamsul Ola M, Moore P, Maddox D, El-Sherbeny A, Huang W, Roon P, Agarwal N, Ganaphthy V, Smith S B. Analysis of sigma receptor (sigmaR1) expression in retinal ganglion cells cultured under hyperglycemic conditions and in diabetic mice. Brain Res Mol Brain Res. 2002 Nov. 15; 107(2):97-107.
Shi, J., J. D. Bui, S. H. Yang, T. H. Lucas, D. L. Buckley. S. P. Blackband, M. A. King, A. L. Day and J. W. Simpkins, Estrogens decrease reperfusion-associated cortical ischemic damage: a MRI analysis in a transient focal ischemia model. Stroke, 32:987-992, 2001.
Shim S S, Grant E R, Singh S, Gallagher M J, Lynch D R. Actions of butyrophenones and other antipsychotic agents at NMDA receptors: relationship with clinical effects and structural considerations. Neurochem Int. 1999 34:167-175.
Simpkins, J. W., G. Rajakumar, Y. Q. Zhang, C. E. Simpkins, D. Greenwald, C. H. Yu, N. Bodor, and A. L. Day, Estrogens may reduce mortality and ischemic damage caused by middle cerebral artery occlusion in the female rat. J. Neurosurg 87: 724-730, 1997.
Simpson L., McNiece I., Newberg M., Schetz J., Lynch K., Quesenberry P., Isakson P. (1989), Detection and characterization of a B cell stimulatory factor (BSF-TC) derived from a bone marrow stromal cell line. J. Immunol., 142:3894-3900.
Su T P, Wu X Z, Cone E J, Shukla K, Gund T M, Dodge A L, Parish D W. Sigma compounds derived from phencyclidine: identification of PRE-084, a new, selective sigma ligand. J Pharmacol Exp Ther. 1991; 259:543-550.
Sumien N, Heinrich K R, Sohal R S, Forster M J. Short-term vitamin E intake fails to improve cognitive or psychomotor performance of aged mice. Free Radic Biol Med. 2004 Jun. 1; 36(11):1424-33.
Takagi K, Ginsberg M D, Globus M Y, Dietrich W D, Martinez E, Kraydieh S, Busto R. Changes in amino acid neurotransmitters and cerebral blood flow in the ischemic penumbral region following middle cerebral artery occlusion in the rat: correlation with histopathology. J Cereb Blood Flow Metab. 1993 July; 13(4):575-85.
Takahashi H, Kirsch J R, Hashimoto K, London E D, Koehler R C, Traystman R J. PPBP [4-phenyl-1-(4-phenylbutyl)piperidine] decreases brain injury after transient focal ischemia in rats. Stroke. 1996 November; 27(11):2120-3.
Takahashi H, Kirsch J R, Hashimoto K, London E D, Koehler R C, Traystman R J. PPBP [4-phenyl-1-(4-phenylbutyl)piperidine], a potent sigma-receptor ligand, decreases brain injury after transient focal ischemia in cats. Stroke. 1995 September; 26(9):1676-82.
Tan S, Wood M, Maher P. Oxidative stress induces a form of programmed cell death with characteristics of both apoptosis and necrosis in neuronal cells. J Neurochem. 1998 July; 71(1):95-105.
Traynelis S F, Lipton S A. Is tissue plasminogen activator a threat to neurons? Nat Med. 2001 January; 7(1):17-8.
Vilner B J, John C S, Bowen W D. Sigma-1 and sigma-2 receptors are expressed in a wide variety of human and rodent tumor cell lines. Cancer Res. 1995 55:408-413.
Wadenberg M L, Kapur S, Soliman A, Jones C, Vaccarino F. Dopamine D2 receptor occupancy predicts catalepsy and the suppression of conditioned avoidance response behavior in rats. Psychopharmacology (Berl). 2000 July; 150(4):422-9.
Wen Y, Yang S, Liu R, Brun-Zinkemagel A M, Koulen P, Simpkins J W. Transient cerebral ischemia induces aberrant neuronal cell cycle re-entry and Alzheimer's disease-like tauopathy in female rats. J Biol. Chem. 2004 279:22684-22692.
Whittemore E R, Ilyin V I, Woodward R M. Antagonism of N-methyl-D-aspartate receptors by sigma site ligands: potency, subtype-selectivity and mechanisms of inhibition. J Pharmacol Exp Ther. 1997 282:326-338.
Willmot M, Gibson C, Gray L, Murphy S, Bath P. Nitric oxide synthase inhibitors in experimental ischemic stroke and their effects on infarct size and cerebral blood flow: a systematic review. Free Radic Biol Med. 2005; 39:412-25.
Wong G Y, Warner D O, Schroeder D R, Offord K P, Warner M A, Maxson P M, Whisnant J P. Risk of surgery and anesthesia for ischemic stroke. Anesthesiology. 2000 February; 92(2):425-32.
Yamamoto H, Miura R, Yamamoto T, Shinohara K, Watanabe M, Okuyama S, Nakazato A, Nukada T. Amino acid residues in the transmembrane domain of the type 1 sigma receptor critical for ligand binding. FEBS Lett. 1999 Feb. 19; 445(1):19-22.
Yang Y, Shuaib A, Li Q. Quantification of infarct size on focal cerebral ischemia model of rats using a simple and economical method. J Neurosci Methods. 1998 Oct. 1; 84(1-2):9-16.
Yang, S. H., Liu, R., Wu, S. S, and Simpkins, J. W., The use of estrogens and related compounds in the treatment of damage from cerebral ischemia, Ann N Y Acad Sci, 1007 (2003) 101-7.
Yang, S.-H., E. Perez, J. Cutright, R. Liu, Z. He, A. L. Day and J. W. Simpkins, Testosterone increases neurotoxicity of glutamate in vitro and ischemia-reperfusion injury in an animal model. J. Applied Physiol. 92: 195-201, 2002.
Yang, S. H., A. L. Day and J. W. Simpkins, Estradiol exerts neuroprotective effects when administrated after ischemic insult. Stoke 31: 745-749, 2000.
Yang, S.-H., Z He, S. S. Wu, Y.-J. He, J. Cutright, W. J. Millard, A. L Day, and J. W. Simpkins, 17 □-estradiol can reduce secondary ischemic damage and mortality of subarachnoid hemorrhage, J Cerebral Blood Flow and Metab. 21: 174-181, 2001.
Yao J K, Leonard S, Reddy R D. Increased nitric oxide radicals in postmortem brain from patients with schizophrenia. Schizophr Bull. 2004; 30(4):923-34.
Yoshida T, Limmroth V, Irikura K, Moskowitz M A. The NOS inhibitor, 7-nitroindazole, decreases focal infarct volume but not the response to topical acetylcholine in pial vessels. J Cereb Blood Flow Metab. 1994 November; 14(6):924-9.
Zhang Z G, Reif D, Macdonald J, Tang W X, Kamp D K, Gentile R J, Shakespeare W C, Murray R J, Chopp M. ARL 17477, a potent and selective neuronal NOS inhibitor decreases infarct volume after transient middle cerebral artery occlusion in rats. J Cereb Blood Flow Metab. 1996 July; 16(4):599-604.
Zhang Q, Sachdev P S. Psychotic disorder and traumatic brain injury. Curr Psychiatry Rep. 2003; 5(3):197-201.
Zhang, Y. Q., J. Shi, G. Rajakumar, A. L. Day, and J. W. Simpkins. Effects of gender and estradiol treatment on focal brain ischemia. Brain Res. 784: 321-324, 1998.
Zhang Z G, Chopp M, Bailey F, Malinski T. Nitric oxide changes in the rat brain after transient middle cerebral artery occlusion. J Neurol Sci. 1995 January; 128(1):22-7.
Zaulyanov L L, Green P S, Simpkins J W. Glutamate receptor requirement for neuronal death from anoxia-reoxygenation: an in Vitro model for assessment of the neuroprotective effects of estrogens. Cell Mol. Neurobiol. 1999 19:705-718.

1-8. (canceled)

9. A method for reducing the effect of ischemia comprising contacting cells with a pharmaceutically effective amount of a butyrophenone, wherein the butyrophenone bind to a sigma-1 receptor and protect the cells from the ischemia.

10. The method of claim 9, wherein the composition is administered several hours before to about 720 minutes after the occurrence of an ischemic cerebral trauma.

11. The method of claim 9, wherein the ischemic injury comprises a cerebral vascular accident, a head trauma or a stroke.

12. The method of claim 9, wherein the composition further comprises a therapeutic agent selected from the group consisting of t-PA, streptokinase, urokinase, aspirin, dipyridamole, a thrombolytic, an antithrombotic drug, combinations and mixtures thereof.

13. The method of claim 9, wherein the one or more butyrophenones are provided at a dose between about 0.5 and 100 mg per day.

14. The method of claim 9, wherein the one or more butyrophenones are provided at a dose between about 0.5 and 20 mg per day.

15. The method of claim 9, wherein the butyrophenone is Haloperidol and metabolites thereof.

16. The method of claim 9, wherein the butyrophenone is provided in an amount sufficient to occupy greater than about 65% of the D2 dopamine receptor in vivo.

17. The method of claim 9, wherein the butyrophenone is adapted for oral, intravenous, subcutaneous, sublingual, intramuscular, intranasal or mucosal or other administration.

18. The method of claim 9, wherein the pharmaceutically effective amount of the antipsychotic butyrophenone at about 0.001 mg/kg to about 10 mg/kg for 0.5 to 2.5 h.

19. The method of claim 9, wherein the composition is adapted for administration to a patient before surgery that will include an ischemic interval.

20-28. (canceled)

29. A method for reducing the effect of ischemia comprising the steps of:
identifying a patient that will undergo an ischemic interval during surgery; and providing the patient a pharmaceutically effective amount of a butyrophenone sufficient to protect the patient from the ischemic interval

30. The method of claim 29, wherein the composition is administered between about one hour before to about 2 weeks after the occurrence of an ischemic cerebral trauma.

31. The method of claim 29, wherein the ischemic injury comprises a cerebral vascular accident, a head trauma or a stroke.

32. The method of claim 29, wherein the composition further comprises a therapeutic agent selected from the group consisting of t-PA, streptokinase, urokinase, aspirin, dipyridamole, a thrombolytic, an antithrombotic drug, combinations and mixtures thereof.

33. The method of claim 29, wherein the butyrophenone is provided at a dose between about 0.05 and 30.0 mg per day.

34. The method of claim 29, wherein the patient is provided the composition before, during, after the surgery and combinations thereof.

35. The method of claim 29, wherein the surgery is selected from general surgery, orthopedic, spinal, coronary artery bypass grafting (CABG), carotid endarterectomy and aneurysms.

36-38. (canceled)

39. A method for reducing the effect of ischemia comprising contacting one or more cells with a pharmaceutically effective amount of one or more compounds selected from Haloperidol and metabolites thereof in an amount sufficient to protect cells from the ischemia.

40-42. (canceled)

43. The method of claim 39, wherein the ischemia comprises a cerebral ischemia or a stroke.

44. The method of claim 39, wherein the ischemia comprises a tissue that is the subject of a surgical procedure that includes an ischemic event.

45. The method of claim 39, wherein the ischemia is during a surgery selected from general surgery, orthopedic, spinal, coronary artery bypass grafting (CABG), carotid endarterectomy and aneurysms.

46. A method of treating a human being suffering from ischemia comprising administering a therapeutically effective amount of a Haloperidol or metabolite thereof wherein the amount is sufficient to protect a cell or tissue from ischemia.

47. (canceled)
